(19) United States
(12) Patent Application Publication
(10) Pub. No.: US 2005/0079176 A1
(43) Pub. Date: Apr. 14, 2005

(54) Treating stress response with chemokine receptor ccr5 modulators

(76) Inventors: Richard N. Pierson III, Ruxton, MD (US); Agnes M. Azimzadeh, Timonium, MD (US); Carsten Schroeder, Baltimore, MD (US); Julie A. DeMartino, Westfield, NJ (US)

Correspondence Address: MERCK AND CO., INC, P O BOX 2000, RAHWAY, NJ, 07065-0907, US

(21) Appl. No.: 10/501,999

(22) PCT Filed: Jan. 22, 2003
(86) PCT No.: PCT/US03/01874

Related U.S. Application Data
(60) Provisional application No. 60/350,868, filed on Jan. 22, 2002.
(60) Provisional application No. 60/365,097, filed on Mar. 18, 2002.

(51) Int. Cl. A61K 39/395; A61K 38/00; A61K 31/00

(52) U.S. Cl. 424/145.1; 514/1; 514/2

(57) ABSTRACT

The use of chemokine receptor CCR5 modulators (e.g., CCR5 antagonists) is disclosed for the treatment or prevention of stress response (e.g., fever) in a subject resulting from surgery, infection or other insult to the subject (e.g., a warm-blooded vertebrate). Methods for treating or preventing disorders involving the activation of pro-inflammatory cytokines by administration of a CCR5 modulator, for inhibiting the endogenous production of pro-inflammatory cytokines by the administration of CCR5 modulators, and for determining the efficacy of CCR5 modulators in correcting abnormal levels of pro-inflammatory cytokines are also disclosed.

This application claims the benefit of U.S. Provisional Application No. 60/350,868, filed Jan. 22, 2002, and U.S. Provisional Application No. 60/365,097, filed Mar. 18, 2002, the disclosures of which are hereby incorporated by reference in their entireties.

### FIELD OF THE INVENTION

The present invention relates generally to therapeutic methods for alleviating stress responses. More particularly, the present invention relates to the administration of a chemokine receptor CCR5 modulator (e.g., a CCR5 receptor antagonist) to treat a subject suffering from a stress response (e.g., fever and malaise as a result of infection and/or injury) or to prevent a stress response in a subject at risk therefor (a pre-operative surgical patient). The present invention also relates to the administration of CCR5 modulators for the treatment or prevention of the inflammatory response mediated by pro-inflammatory cytokines.

### BACKGROUND OF THE INVENTION

Physical injury, wounds, surgery, burns, infection, and other insults to the body typically result in a stress response that can include fever, pain, headache, inflammation, malaise, and/or other conditions. The stress response can sometimes be beneficial in that it can be part of the body's immune response to the insult and a tool in the restoration of health. For example, fever in response to a bacterial infection typically will retard the growth of the bacterial invader and at the same time increase the bactericidal activity of neutrophils and macrophages responding to the infection (Netea et al., Clinical Infectious Diseases 2000, 31: S178-S184). Unfortunately, the body's response to an insult is in many circumstances harmful, leading to dangerously high fever, severe inflammation, tissue destruction, etc., which can impede recovery and even lead to shock or death. The fever and malaise that typically follow surgery, for example, can cause significant post-operative discomfort without contributing to recovery.

Physical injury, surgery, infection, and other traumatic insults, as well as a variety of other immunological disorders, provoke a biochemical cascade of pathophysiologic events that is triggered by excessive tissue or blood concentrations of pro-inflammatory cytokines released or synthesized following the insult. The pro-inflammatory cytokine, interleukin-1 (IL1), is at the head of many inflammatory cascades and its actions, often via the induction of other cytokines, lead to activation and recruitment of leukocytes into damaged tissue, local production of vasoactive agents, and hepatic acute phase response. That it is a key mediator in the inflammatory response is evident from the description set forth in Dinarello, Blood 1991, 77: 1627-1652; Dinarello et al., New England J. Med. 1993, 328: 106-113; and Dinarello FASEB J. 1994, 8:1314-1325.

IL1 is produced by a number of cell types, including monocytes and macrophages. When locally released, IL1 diffuses into the circulation, where it is ultimately carried to the hypothalamus. There, it acts to stimulate the production of prostaglandin-E which acts as an inflammatory mediator. IL1 has also been shown to be an endogenous human pyrogen, which produces fever (Ikejima et al., J. Clin. Invest. 1984, 73: 1312). Two forms of IL1 have been isolated, IL1-α and IL1-β. IL1 is known to incite a variety of other systemic responses; e.g., it mobilizes neutrophils, stimulates liver production of acute phase proteins and complements, and is also responsible for the increases in circulating eicosanoid levels, levels of interleukin-6 and levels of tumor necrosis factor (Dinarello, Rev. Infect. Disease, 6: 51-94).

Interleukin-6 (IL6) is a multi-functional cytokine that plays a pivotal role in host defense mechanisms (Heinrich et al., Biochem. J. 1990, 265: 621; Van Snick, J. Annu. Rev. Immunol. 1990, 8: 253; and Hirano et al., Immunol. Today 1990, 11: 443). However, examples of disorders characterized by elevated serum levels of IL6 in patients abound, and overproduction of IL6 has been implicated in sequellae of transplantations, autoimmune diseases and, in particular, certain types of septicemia. Indeed, the overproduction of IL6 has been suggested to play a role in the pathogenesis of the above referenced diseases (Hirano et al., Immunol. Today 1990, 11: 443; Sehgal, Proc. Soc. Exp. Biol. Med. 1990, 195: 183; Grau, Eur. Cytokine Net 1990, 1: 203; Bauer et al., Ann. Hematol. 1991, 62: 203; Campbell et al., J. Clin. Invest. 1991, 7: 739; and Roodman et al., J. Clin. Invest. 1992, 89: 46).

IL6 induction rapidly follows injury or other insult. Plasma levels of IL6 can be detected as early as 30 minutes after incision in patients undergoing elective surgery (Shenkin et al., Lymphok. Res. 1989, 8: 123-127). Maximal levels of IL6 are found between 90 minutes and 6 hours post surgery (Pullicino et al., Lymphok. Res. 1990, 9: 2-6; Shenkin, Lymphok. Res. 1989, 8: 123-127). In contrast, upon exposure to an infectious agent, elevated plasma levels may persist for days (Bauer, J. et al., Ann. Hematol. 1991, 62: 203-210). Importantly, elevated serum levels of IL6 have been observed in transplant rejection and inflammatory bowel disease (van Oers et al., Clin. Exper. Immunol. 1988, 71: 314-319; Bauer et al., Ann. Hematol. 1991, 62: 203-210).

While numerous approaches to regulate the production of interleukin-6 have been proposed, no substance or method has been reported to inhibit specifically the production of IL6 or to effectively block its adverse actions. In fact, no natural receptor-antagonist for IL6 has so far been identified.

The tumor necrosis factor (TNF) family is an expanding set of extracellular signaling molecules (ligands) with biological activities that are intimately associated with a variety of disease conditions. There are several disease states in which excessive or unregulated TNF production by monocytes, macrophages or related cells are implicated in exacerbating and/or causing the disease. For example, TNF, the prototypic member of this family, is well known as a mediator of septic shock, inflammation, and graft-versus-host disease. (See, for example, Cerami, Immunol Today 1988, 9: 28-31; Revel, Ciba Found. Symp. 1987, 129: 223-233; Cohen, J. Bone Marrow Transplant. 1988, 3(3): 193-197).

Septic shock is a life-threatening complication of bacterial infections. It results from the uncontrolled, sequential release of mediators having pro-inflammatory activity following infection with gram-negative bacteria, and in response to endotoxins (see, e.g., Tracey et al, Science 1986, 234: 470; Alexander et al, J. Exp. Med. 1991, 173: 1029; Doherty et al, J. Immunol. 1992, 149: 1666; Wysocka et al., Eur. J. Immunol. 1995, 25: 672). Endotoxin exerts its effect by inducing potent macrophage activation, and release of cytokines such as TNF-α, IL1, IL6, IL12, and interferon-gamma (IFN-γ) (see Van Deuren et al, J. Pathol. 1992, 168: 349). Two key mediators of septic shock are TNF and IL1, which are released by macrophages and appear to act synergistically to cause a cascade of physiological changes leading to circulation collapse and organ failure (Bone, Ann. Intern. Med. 1991, 115: 457-469). Overproduction of IL6 has also been linked to septic shock (Starnes, Jr., et al., J. Immunol. 1990, 145: 4185). The central role of pro-inflammatory cytokines in the pathogenesis of septic shock (also referred to as endotoxic shock) is underlined by the occurrence of high levels of circulating cytokines in both humans and experimental animals during endotoxemia (see Stevens et al., Curr. Opin. Infect. Dis. 1993, 6: 374). Significantly, a substantial body of literature shows that anti-cytokine action can improve the outcome of subjects challenged by endotoxin or gram-negative bacteria (see Beutler et al., Science 1985, 229: 689, and Heinzel et al., J. Immunol. 1990, 145: 2920). For example, Bozza et al., J. Exp. Med. 1999, 189: 341 teaches targeting of genes encoding pro-inflammatory cytokines, and Ohlsson et al., Nature 1990, 348: 550 teaches administration of IL1 receptor antagonists.

Pro-inflammatory cytokines including IL1, IL6 and TNF mediate the condition known as sepsis in substantially the same manner as septic shock. Sepsis—also known as septicemia, septic syndrome and septic response—has no standard definition, but typically refers to severe systemic infection. The traditional agents for sepsis were gram-negative bacteria, but more recently patients have been observed with characteristic responses of sepsis without a clearly identifiable inciting microbe. The term sepsis has thus come to be associated with any systemic response to overwhelming infection or other severe insult (Kelly et al, Ann. Surg. 1997, 225(5): 530-541, see esp. 542-543). Despite the major advances of the past several decades in the treatment of serious infections, sepsis remains a serious health threat (S. M. Wolff, New Eng. J. Med. 1991, 324: 486-488).

The foregoing observations have buttressed the importance of regulating pro-inflammatory cytokine production for the maintenance of the homeostasis of immune system in a human body and for the treatment and prophylaxis of pathologies attending a post surgical inflammatory response. Thus there remains a need for an effective, clinically applicable approach for preventing or treating a post-surgical stress response/inflammatory response and cytotoxic T-lymphocyte (CTL) and/or complement-dependent rejection of organ or tissue transplants.

Stress response accompanying physical injury, surgery (including tissue and organ transplantation), infection and other insults has been treated with some success with various drugs. Corticosteroids and immunosuppressants are often employed, for example, but these agents have serious, well known side effects. Non-steroidal antiinflammatory drugs (NSAIDs) are also employed, but they are known to cause gastric ulceration. There is a need for new drugs for the therapeutic or prophylactic treatment of stress response. More particularly, there is a need for agents that will reduce or suppress the activation of inflammatory cells or their production of pro-inflammatory cytokines in response infection (especially serious and/or systemic infection), major surgery, allograft transplant rejection, physical injury, and other traumatic insults.

The following references are of interest as background:

WO 00/76972 discloses N-cyclopentyl modulators of the activity of chemokine receptors including CCR5.

WO 01/78707 discloses a method of treating the rejection of transplanted grafts by administration of an antagonist of CCR5 function to the graft recipient.

Leon, J. Appl. Physiol. 2002, 92: 2648-2655 is a review of the cytokine regulation of fever that discusses the role of IL1, IL6, TNF-α, and IL10 in the inducement and inhibition of fever.

### SUMMARY OF THE INVENTION

The present invention is directed to the use of chemokine receptor CCR5 modulators (e.g., CCR5 antagonists) to treat or prevent stress response (e.g., fever) in a subject resulting from a planned (e.g., surgery) or unforeseen (e.g., injury due to an accident) insult to the subject. The present invention also includes a method for treating or preventing a disorder involving the activation of pro-inflammatory cytokines by administration of a CCR5 modulator. The present invention further includes a method for inhibiting the endogenous production of pro-inflammatory cytokines by the administration of CCR5 modulators. The present invention still further includes a method for determining the efficacy of CCR5 modulators in correcting abnormal levels of pro-inflammatory cytokines.

Other embodiments, aspects and features of the present invention are either further described in or will be apparent from the ensuing description, examples and appended claims.

### DETAILED DESCRIPTION OF THE INVENTION

The present invention includes methods for treating or preventing a stress response via administration of a therapeutically effective amount of a chemokine receptor CCR5 modulator to a subject suffering from a stress response or at risk to suffer from a stress response. More particularly, the present invention includes a method of treating or preventing stress response in a subject in need thereof, which comprises administering a therapeutically effective amount of a CCR5 antagonist to the subject. Treatment or prevention of the stress response can hasten the subject's return to normal activity with a reduced requirement for narcotic analgesics and/or with a lower complication rate. In an embodiment of this method, the subject is a warm-blooded vertebrate. In an aspect of this embodiment, the subject is a primate, and is especially a human. In another embodiment of the method, the subject is a surgical patient who has pre-existing infection (e.g., sepsis from abscess or empyema) or inflammation (e.g, rheumatoid arthritis or acute myocardial infarction). In still another embodiment of the method, the subject is a cardiac surgery patient. In an aspect of this embodiment, the cardiac surgery patient is a patient who has recently experienced a myocardial infarction or who has a lung infection or liver disease.

Another embodiment of this method is a method of treating or preventing stress response in a subject in need thereof, which comprises administering a therapeutically effective amount of a CCR5 antagonist to the subject, wherein the stress response comprises inflammation and associated pain and/or malaise resulting or expected from a planned stress. In an aspect of this embodiment, the planned stress is surgery. The surgery can be any surgical procedure including, but not limited to, major surgery such as cardiac surgery, a minor outpatient procedure, and minimal access surgery (also known in the art as minimally invasive surgery) such as laparoscopy or thoracoscopy. The method can reduce or preclude the delay in the return of normal respiratory and bowel function that is often observed in surgical patients.

As used herein, the term “stress response” refers to any response (i.e., physiological change) seen in a subject exposed to an insult (which may alternatively be referred to as a stressor). An insult is a trauma (e.g., physical injury, wounds, surgery, burns) or a physiopathological state (e.g., infection such as bacteremia, endotoxin infusion) that results in changes to existing rhythmical processes which are homeostatic in nature. Stress response includes, but is not limited to, any one or more of the following conditions: hyperthermia, hypothermia, hypertension, hypotension, inflammation, malaise (i.e., discomfort or debility typically characterized by decreased activity and/or loss of appetite), shock (e.g., septic shock), tissue damage, organ damage and/or failure, and sepsis. Fever and malaise, for example, are typically seen in mammals after organ transplantation using conventional immunosuppression.

The term “treating”, or a variant thereof (e.g., “treatment”), refers to reducing or ameliorating an existing undesirable or adverse condition, symptom or disease (e.g., stress response due to exposure to a stressor) or delaying its onset in a subject in need of such reduction, amelioration or delay.

The term “preventing”, or a variant thereof (e.g., “prevention”), refers to prophylaxis of an undesirable or adverse condition, symptom or disease in a subject who is at increased risk of acquiring such a condition, symptom, or disease as a result of being subjected or exposed to an insult. “Increased risk” means a statistically higher frequency of occurrence of the condition, symptom, or disease in the subject as a result of the insult in comparison to the frequency of its occurrence in the general population (e.g., an individual about to have surgery would be at a substantially increased risk for hyperthermia and inflammation subsequent to the surgery).

The term “subject” as used herein refers to any vertebrate species which is the object of treatment, observation or experiment with respect to the present invention. In one embodiment, the subject is a warm-blooded vertebrate, particularly a mammal, preferably a primate, and more preferably a human. A mammal is understood to include any mammalian species in which treatment or prevention is necessary or desirable, particularly agricultural and domestic mammalian species. Thus, subjects contemplated for the present invention include primates (including humans), as well as those mammals of importance due to being endangered (such as Siberian tigers), of economic importance (animals raised on farms for human consumption) and/or social importance (animals kept as pets or in zoos) to humans, such as cats, dogs, swine (e.g., pigs, hogs, and wild boars), ruminants (e.g., cattle, oxen, sheep, giraffes, deer, goats, bison, and camels), and horses. Birds are also contemplated as subjects in the present invention including birds that are endangered, kept in zoos, as well as fowl, and more particularly domesticated fowl (e.g., poultry, such as turkeys, chickens, ducks, geese, guinea fowl, and the like) of economic importance to humans.

The term “patient” refers to a subject as defined above who/which is awaiting or receiving medical care or is or will be the object of a medical procedure (e.g., surgery).

The term “cardiac surgery patient” refers to a patient who has or will have open heart surgery using cardiopulmonary bypass. “Cardiopulmonary bypass”, or a variant thereof (e.g., “bypass” or “circulatory bypass”) refers to circulatory bypass of the heart and lungs; i.e., the condition wherein the heartbeat is stopped for the purpose of surgery on the still heart, and the blood supply to the brain and the remainder of the body, excluding the heart and lungs, is provided by an extracorporeal machine that oxygenates and pumps the blood.

The term “transplant” refers to the grafting, implantation or transplantation of organs, tissues, cells (e.g., bone marrow) and/or biocompatible materials onto or into the body of an animal. The term encompasses the transfer of tissues from one part of the animal's body to another part and the transfer of organs, tissues, and/or cells obtained from a donor animal (either directly or indirectly such as an organ or tissue produced in vitro by culturing cells obtained from the animal) into a recipient animal. The animal is suitably a warm-blooded vertebrate, is typically a mammal, and is especially a primate (e.g., a human). The term “transplant rejection” means any immune reaction in the recipient directed against grafted organs, tissues, cells, and/or biocompatible materials.

The term “therapeutically effective amount” (or more simply an “effective amount”) as used herein means that amount of active agent or active ingredient (e.g., chemokine receptor CCR5 modulator, especially a CCR5 antangonist) that elicits the biological or medicinal response in a tissue, system, animal or human that is being sought by a researcher, veterinarian, physician or other clinician, which includes alleviation or prophylaxis of the symptoms of the disease or condition being treated or prevented. When the salt of a chemical compound is administered, references to the amount of active ingredient are to the free acid or free base form of the compound. Actual dosage levels of active ingredients in a composition employed in a method of the present invention can be varied so as to administer an amount of the active compound(s) that is effective to achieve the desired therapeutic response for a particular subject and/or application.

The term “administration”, or a variant thereof (e.g., “administering”), means providing the active agent or active ingredient (e.g., a CCR5 antagonist), alone or as part of a pharmaceutically acceptable composition, to the subject (e.g., warm-blooded vertebrate) in whom/which the condition, symptom, or disease is to be treated or prevented.

By “pharmaceutically acceptable” is meant that the ingredients of a pharmaceutical composition are compatible with each other and not deleterious to the recipient thereof.

The present invention also includes a method of treating or preventing hyperthermia in a subject in need thereof, which comprises administering a therapeutically effective amount of a CCR5 antagonist to the subject. Embodiments of this method include the method as just described wherein the subject is a warm-blooded vertebrate, or is a primate, or is a human. Other embodiments of this method include the method as originally described wherein the subject is other than a graft transplant patient, or is a cardiac surgery patient.

The present invention also includes a method of treating or preventing hypothermia in a subject in need thereof, which comprises administering a therapeutically effective amount of a CCR5 antagonist to the subject. Embodiments of this method include the method as just described wherein the subject is a warm-blooded vertebrate, or is a primate, or is a human. Other embodiments of this method include the method as originally described wherein the subject is other than a graft transplant patient, or is a cardiac surgery patient.

The term “hyperthermia” refers herein to the elevation of the temperature of a subject's body, or a part of a subject's body, compared to the normal temperature of the subject. In mammals, a normal body temperature is ordinarily maintained due to the thermoregulatory center in the anterior hypothalamus, which acts to balance heat production by body tissues with heat loss. The terms “fever” and “hyperthermia” are sometimes distinguished from each other, wherein fever refers to a regulated elevation in a subject's thermal set point (in response, e.g., to an infection or other insult), and hyperthermia refers to an unregulated rise in body temperature that is not triggered by an increased thermal set point but is instead in response to an internal (e.g., exercise) or external (e.g., hot ambient conditions) source of heat. The terms “fever” and “hyperthermia” are used interchangeably herein, and both refer to a regulated rise in body temperature in response to an insult or other inflammatory stimulus.

The term “hypothermia” refers to a decrease in the temperature of a subject's body, or a part of a subject's body, compared to the normal temperature of the subject. The decrease is typically a regulated decrease in the subject's thermal set point, such as in response to an insult (e.g., infection).

Chemokines are a family of pro-inflammatory mediators that promote recruitment and activation of multiplelineages of leukocytes (e.g., lymphocytes, macrophages). They can be released by many kinds of tissue cells after activation. Continuous release of chemokines at sites of inflammation can mediate the ongoing migration and recruitment of effector cells to sites of chronic inflammation. The chemokines are related in primary structure and share four conserved cysteines, which form disulfide bonds. Based upon this conserved cysteine motif, the family can be divided into distinct branches, including the C—X—C chemokines (a-chemokines), and the C—C chemokines (P-chemokines), in which the first two conserved cysteines are separated by an intervening residue, or are adjacent residues, respectively (Baggiolini, M. et al., Immunology Today 1994, 15: 127-133).

The C—X—C chemokines include a number of potent chemoattractants and activators of neutrophils, such as interleukin 8 (IL-8), PF4 and neutrophil-activating peptide-2 (NAP-2). The C—C chemokines include, for example, RANTES (Regulated on Activation, Normal T Expressed and Secreted), macrophage inflammatory proteins 1-alpha and 1-beta (MIP-1α and MIP-1β), eotaxin and human monocyte chemotactic proteins 1 to 3 (MCP-1, MCP-2, MCP-3), which have been characterized as chemoattractants and activators of monocytes or lymphocytes. Chemokines, such as IL-8, RANTES and MIP-1α, for example, have been implicated in human acute and chronic inflammatory diseases including respiratory diseases, such as asthma and allergic disorders.

The chemokine receptors are members of a superfamily of G protein-coupled receptors (GPCR) which share structural features that reflect a common mechanism of action of signal transduction (Gerard, C. et al., Annu Rev. Immunol. 1994, 12: 775-808; Gerard, C. et al., Curr. Opin. Immunol. 1994, 6: 140-145). Conserved features include seven hydrophobic domains spanning the plasma membrane, which are connected by hydrophilic extracellular and intracellular loops. The majority of the primary sequence homology occurs in the hydrophobic transmembrane regions with the hydrophilic regions being more diverse. The receptors for the C—C chemokines include: CCR1 which can bind, for example, MIP-1α, RANTES, MCP-2, MCP-3, MCP-4, CKbeta8, CKbeta8-1, leukotactin-1, HCC-1 and’ MPIF-1; CCR2 which can bind, for example, MCP-1, MCP-2, MCP-3 and MCP-4; CCR3 which can bind, for example, eotaxin, eotaxin-2, RANTES, MCP-2, MCP-3 and MCP-4; CCR4 which can bind, for example, TARC or MDC; CCR5 which can bind, for example, MIP-1α, RANTES, MIP-1β, MCP-1, MCP-2 and MCP-4; CCR6 which can bind, for example, LARC/MIP-3α/exodus; CCR7 which can bind, for example, ELC/MIP-3β; CCR8 which can bind, for example, I-309; CCR9 which can bind, for example, TECK; and CCR10 which can bind, for example, ESkine and CCL27 (Baggiolini, M., Nature 1998, 392: 565-568; Luster, A. D., New England J. Med. 1998, 338(7): 436-445; Tsou et al., J. Exp. Med. 1998, 188: 603-608; Nardelli et al., J. Immunol. 1999, 162(1): 435-444; Youn et al., Blood 1998, 91(9): 3118-3126; Youn, et al., J. Immunol. 1997, 159(11): 5201-5205; Zaballos et al., J. Immunol. 1999, 162: 5671-5675; Jannin et al., J. Immunol. 2000, 164: 3460-3464; Homey et al., J. Immunol. 2000, 164: 3465-3470). The receptors for the CXC chemokines include: CXCR1 which can bind, for example, IL-8, GCP-2; CXCR2 which can bind, for example, IL-8, GROalpha/beta/gamma, NAP-2, ENA78, GCP-2; CXCR3 which can bind, for example, interferon gamma (IFNγ)-inducible protein of 10 kDa (IP-10), monokine induced by IFNγ (Mig), interferon-inducible T cell chemoattractant (I-TAC); CXCR4 which can bind, for example, SDF-1; and CXCR5 which can bind, for example, BCA-1/BLC (Baggiolini M., Nature 1998, 392: 565-568; Lu et al., Eur. J. Immunol. 1999, 29: 3804-3812).

An aspect of the present invention is that blockade of hyperthermic response to stress (e.g., surgical stress and/or graft ischemia/reperfusion injury) and of hypothermic response to stress can be provided by CCR5 inhibition. The term “inhibition” (or “inhibiting”) refers to the reduction or suppression of a given condition, symptom, or disease, wherein in this case the condition is due to the activity of the CCR5 receptor. While not wishing to be bound by any particular theory of operation, it is believed that CCR5 and associated chemokines (MIP-1α, MIP-1β and RANTES) mediate the cytokine-driven innate immune response to stress. As described more fully in Example 1 below, studies of the stress response in monkeys following cardiac allotransplantation have been performed. In the studies, twelve monkeys received cardiac allografts. Five were treated with a CCR5 antagonist (only) beginning at transplant, two were treated with a CCR5 antagonist combined with cyclosporin beginning at transplant, and three received only saline infusion (“control”). Two animals were treated with cyclosporin only. Six out of the seven monkeys treated with a CCR5 antagonist did not develop a fever (i.e., a temperature greater than 38.5° C.) while recovering from the transplantation procedure, and had an average temperature over the first three days after surgery (circa 37.5° C.) that was about one degree lower than the control monkeys (ca. 38.5° C.). The average temperature in the CCR5 antagonist-treated animals was about 0.5° C. lower than in the cyclosporin-only-treated animals (ca. 38.0° C.). The CCR5 antagonist-treated monkeys did not exhibit malaise and behaved as if they had not had surgery. The CCR5 antagonist-treated monkeys also did not exhibit typical symptoms of abdominal tenderness, which would otherwise have been expected, either initially after transplant, or subsequently during rejection of the graft. Absence of malaise and graft abdominal tenderness was unexpected, and was observed despite the occurrence of major biochemical perturbations (i.e., increased creatinine and bilirubin) that normally have been associated with malaise. In one instance these biochemical perturbations resolved during ongoing therapy in an animal whose rejected heart was removed, indicating that the biochemical perturbations were not due to the CCR5 antagonist but rather were due to transplant rejection. The animals also recovered more quickly from surgery as judged by how quickly they resumed normal activity levels, despite performance of multiple additional procedures on days 4 and 7-8 after the initial transplant.

Other agents (e.g., steroids, which block NFkB; non-steroidal anti-inflammatory drugs such as acetaminophen, aspirin (NFkB+COX inhibition), COX-inhibitors) block some consequences of inflammation such as fever. However, they do not reliably prevent important sequellae, such as local and systemic capillary leak, cellular infiltration of tissues, localized pain and systemic malaise, and transient impairment of organ function. Thus, CCR5 blockade can be a useful adjunctive or alternative therapy to steroidal or non-steroidal anti-inflammatory agents for reducing pain, suffering, and inhibition of organ function (e.g., lung and bowel function) associated with the stress of surgery (including major surgery and minimal access surgery), trauma other than surgery (e.g., burns or physical injury), or acute illness, and for control of inflammation in general medical practice.

An aspect of the present invention is that a CCR5 modulator (e.g., a CCR5 antagonist) can ameliorate or block fever (hyperthermia), such as fever resulting from the innate immune response, or hypothermia. While not wishing to be bound by any particular theory of operation, it is believed that CCR5 antagonists can reduce or suppress the elaboration of mediators of inflammation, especially the pro-inflammatory cytokines IL1, IL6 and TNF and most especially IL1 and IL6, and thereby reduce or block fever (or hypothermia) and other symptoms associated with their release in response to an insult.

The present invention also includes a method for treating or preventing stress response in a subject in need thereof, which comprises administering to the subject a CCR5 antagonist in an amount effective to inhibit endogenous production of one or more pro-inflammatory cytokines selected from the group consisting of IL1, IL6, and TNF (e.g., one or more cytokines selected from the group consisting of IL1 and IL6). An embodiment of this method is the method as just described, except that the stress response is stress response to surgery. Another embodiment of this method is the method as originally described, except that the stress is hyperthermia, and is especially surgical hyperthermia (i.e., hyperthermia which arises as a result of surgery). In an aspect of each of the foregoing embodiments, the subject is other than a graft transplant patient. Another embodiment of this method is the method as originally described, except that the stress is hypothermia, such as surgical hypothermia (i.e., hypothermia which arises as a result of surgery).

References herein to IL1, IL6 and TNF are understood to include the various isoforms of each of the cytokines; e.g., IL1-α., IL1-β, TNF-α, and TNF-β. Thus, for example, inhibition of the endogenous production of IL1 is understood to include inhibition of either one or both of its isoforms.

The term “inhibiting the endogenous production” of one or more of the cytokines IL1, IL6 and TNF means: (a) decreasing excessive in vivo levels of the cytokine in the subject (e.g., human) to normal levels for several types of cells, including but not limited to monocytes and/or macrophages; (b) down regulating in the subject's tissue (e.g., human tissue) an excessive in vitro or in vivo level of the cytokine to normal level; or (c) down regulating the cytokine to a normal level by reducing or suppressing direct synthesis of the cytokine as a post-translation event.

The normal level of cytokine can vary from one subject to the next (see, e.g., Roth-Isigkeit A. et al., Clin. Exp. Immunol. 2001, 125: 80-88). Accordingly, in the case of a planned insult such as surgery, it is preferred to determine the normal cytokine level for the given subject prior to the insult. As an alternative to determining the normal cytokine level for the particular subject, the normal level can be equated to the average value obtained or known for a group of similarly situated healthy individuals (i.e., a group of healthy individuals having the same or similar physical condition—age, gender, weight, diet, etc.—and medical history). This alternative approach may be necessary when the normal cytokine level for the given subject cannot be pre-determined (e.g., the individual has been subjected to an unforeseen insult such as a physical injury resulting from an accident) and is not otherwise available in the subject's medical history. Cytokine levels are typically determined in vitro using a sample of the subject's blood. Cytokine levels can be determined, for example, in accordance with the methods described in Casey et al., Ann. Intern. Med. 1993, 119(8): 771-778, and in Bolke et al. Shock 2001, 16(5): 334-9.

A proinflammatory property of IL6 is its ability to stimulate prostaglandin synthesis. Impaired febrile responses are evident in mice which lack the prostaglandin E2 receptor subtype EP3. Consequently, the efficacy of the administration of a CCR5 antagonist in treating or preventing a stress response (e.g., a post-surgical febrile response) can be determined by monitoring the level of production of prostaglandin E2 (PGE2). Accordingly, the present invention also includes a method for treating or preventing stress response (including, e.g., febrile response) in a subject in need thereof, which comprises administering to the subject a CCR5 antagonist in an amount effective to inhibit endogenous production of prostaglandin E2. PGE2 levels can be measured in accordance with the method described in Brideau et al., Inflamm. Res. 1996, (45): 68-74.

In the present invention, the chemokine modulators (e.g., CCR5 antagonists) can be administered with one or more other anti-inflammatory agents, including blockers of other chemokine receptor pathways, steroids, and non-steroidal anti-inflammatory agents. This approach can be viewed as an alternative to anti-TNF, soluble TNF-receptor compounds, or other anti-cytokine agents (IL1, IL6, etc.), none of which alone are believed to provide the effect observed in accordance with the present invention. When the chemokine modulator is administered with another agent (i.e., co-administration), it is understood that the modulator can be administered before, concurrently with, or after administration of the other agent. When administered concurrently, the modulator and the agent can be administered separately at the same time or together in one composition.

Chemokine receptor CCR5 modulators are used in the present methods for modulating chemokine receptor CCR5 activity in tissues, including modulating stress responses in tissues. Thus, as used herein, the terms “modulate”, “modulating”, and “modulator” are meant to be construed to encompass inhibiting, blocking, promoting, stimulating, agonizing, antagonizing, or otherwise affecting chemokine receptor CCR5 activity in tissues.

Such modulators can take a variety of forms that include compounds that interact with the chemokine receptor CCR5 in a manner such that functional interactions with natural chemokine receptor ligands are mimicked, stimulated and/or inhibited. Exemplary modulators include analogs of a chemokine receptor natural ligand binding site on a chemokine receptor CCR5, mimetics of a natural ligand of a chemokine receptor that mimic the structural region involved in chemokine receptor-receptor ligand binding interactions, polypeptides having a sequence corresponding to the domain of a natural ligand of a chemokine receptor, and antibodies which immunoreact with either a chemokine receptor or the natural ligand, all of which exhibit modulator activity as defined herein.

Small organic molecules which are chemokine receptor CCR5 modulators, especially CCR5 antagonists, are suitable for use in the methods of the present invention. Thus, the present invention includes a method of treating or preventing stress response which comprises administering to a subject in need of such treatment a therapeutically effective amount of a compound of Formula (I), or a pharmaceutically acceptable salt or an individual diastereomer thereof: 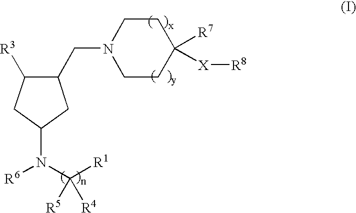
wherein:
X is selected from: —(C₀₋₆ alkyl)-Y—(C₀₋₆ alkyl)-,
   —(C₀₋₆ alkyl)-C₃₋₈ cycloalkyl-(C₀₋₆ alkyl)-,
   C₂₋₁₀ alkenyl, and C₂₋₁₀ alkynyl,
   where the alkyl is unsubstituted or substituted with 1-7 substituents where the substituents are independently selected from:
   (a) halo,
   (b) hydroxy,
   (c) —O—C₁₋₃ alkyl, and
   (d) trifluoromethyl,
and where Y is selected from:
   a single bond, —O—, —SO₂—, —NR¹⁰—, —NR¹⁰—SO₂—, —SO₂—NR¹⁰—, —S—, and —SO—,
and where R¹⁰ is independently selected from: hydrogen, C₁₋₆ alkyl, C₂₋₆ alkenyl, C₂₋₆ alkynyl, C₅₋₆ cycloalkyl, benzyl, phenyl, and C₁₋₆ alkyl-C₃₋₆ cycloalkyl,
   which is unsubstituted or substituted with 1-3 substituents where the substituents are independently selected from: halo, C₁₋₃ alkyl, C₁₋₃ alkoxy and trifluoromethyl;
R¹ is selected from:
   (1) —CO₂H,
   (2) —NO₂,
   (3) -tetrazolyl,
   (4) -hydroxyisoxazole,
   (5) —SO₂NHCO—(C₀₋₃ alkyl)-R⁹, wherein R⁹ is independently selected from: hydrogen, C₁₋₆ alkyl, C₅₋₆ cycloalkyl, benzyl or phenyl, which is unsubstituted or substituted with 1-3 substituents where the substituents are independently selected from: halo, C₁₋₃ alkyl, C₁₋₃ alkoxy and trifluoromethyl, and
   (6) —P(O)(OH)₂;
R³ is selected from the group consisting of:
   phenyl and heterocycle,
      which is unsubstituted or substituted with 1-7 substituents where the substituents are independently selected from:
      (a) halo,
      (b) trifluoromethyl,
      (c) hydroxy,
      (d) C₁₋₃ alkyl,
      (e) —O—C₁₋₃ alkyl,
      (f) —CO₂R⁹,
      (g) —NR⁹R¹⁰, and
      (h) —CONR⁹R¹⁰;
R⁴, R⁵ and R⁶ are independently selected from:
   hydrogen, C₁₋₁₀ alkyl, C₃₋₈ cycloalkyl, C₂₋₁₀ alkenyl,
   C₂₋₁₀ alkynyl, phenyl, —(C₁₋₆ alkyl)-phenyl,
   —(C₁₋₆ alkyl)-C₃₋₈ cycloalkyl, naphthyl, biphenyl, and heterocycle,
      which is unsubstituted or substituted with 1-7 of R¹¹ where R¹¹ is independently selected from:
      (a) halo,
      (b) trifluoromethyl,
      (c) hydroxy,
      (d) C₁₋₃ alkyl,
      (e) —O—C₁₋₃ alkyl,
      (f) —CO₂R⁹,
      (g) —NR⁹R¹⁰, and
      (h) —CONR⁹R¹⁰,
or where R⁴ and R⁵ may be joined together to form a 3-8 membered saturated ring which may be unsubstituted or substituted with 1-7 of R¹¹,
or where R⁵ and R⁶ may be joined together to form a 3-8 membered saturated ring which may be unsubstituted or substituted with 1-7 of R¹¹;
R⁷ is selected from:
   (1) hydrogen,
   (2) C₁₋₆ alkyl, which is unsubstituted or substituted with 1-4 substituents where the substituents are independently selected from: hydroxy, cyano, and halo,
   (3) hydroxy, and
   (4) halo;
R⁸ is selected from:
   hydrogen, C₃₋₈ cycloalkyl, phenyl, naphthyl, biphenyl, and heterocycle,
      which is unsubstituted or substituted with 1-7 of R¹² where R¹² is independently selected from:
      (a) halo,
      (b) cyano,
      (c) hydroxy,
      (d) C₁₋₆ alkyl, which is unsubstituted or substituted with 1-5 of R¹³ where R¹³ is independently selected from: halo, cyano, hydroxy, C₁₋₆ alkoxy, —CO₂H, —CO₂(C₁₋₆ alkyl), trifluoromethyl, and —NR⁹R¹⁰,
      (e) —O—C₁₋₆ alkyl, which is unsubstituted or substituted with 1-5 of R¹³,
      (f) —CF₃,
      (g) —CHF₂,
      (h) —CH₂F,
      (i) —NO₂,
      (j) C₀₋₆ alkyl-phenyl or C₀₋₆ alkyl-heterocycle, which is unsubstituted or substituted with 1-7 substituents where the substituents are independently selected from:
         (i) halo,
         (ii) hydroxy,
         (iii) C₁₋₆ alkyl, unsubstituted or substituted with 1-5 substituents, each of which is independently selected from halo, cyano, hydroxy, C₁₋₆ alkoxy, —CO₂H, —CO₂(C₁₋₆ alkyl), trifluoromethyl, and —NR⁹R¹⁰,
         (iv) —O—C₁₋₆ alkyl,
         (v) —CF₃,
         (vi) —OCF₃,
         (vii) —NO₂,
         (viii) —CN,
         (ix) —SO₂—C₁₋₆ alkyl,
         (x) —CO₂R⁹,
         (xi) —NR⁹R¹⁰,
         (xii) —CONR⁹R¹⁰,
         (xiii) —SO₂—NR⁹R¹⁰,
         (xiv) —NR⁹—SO₂—R¹⁰,
         (xv) —C₃₋₈ cycloalkyl,
         (xvi) —OC₃₋₈ cycloalkyl, and
         (xvii) phenyl;
      (k) —CO₂R⁹,
      (l) tetrazolyl,
      (m) —NR⁹R¹⁰,
      (n) —NR⁹—COR¹⁰,
      (o) —NR⁹—CO₂R¹⁰,
      (p) —CO—NR⁹R¹⁰,
      (q) —OCO—NR⁹R¹⁰,
      (r) —NR⁹CO—NR⁹R¹⁰,
      (s) —S(O)ₘ—R⁹,wherein m is an integer selected from 0, 1 and 2,
      (t) —S(O)₂—NR⁹R¹⁰,
      (u) —NR⁹S(O)₂—R¹⁰,
      (v) —NR⁹S(O)₂—NR⁹R¹⁰,
      (w) C₁₋₆ alkyl substituted with —C₃₋₈ cycloalkyl, and
      (x) —C₃₋₈ cycloalkyl;
n is an integer selected from 1, 2, 3 and 4;
x is an integer selected from 0, 1 and 2, and y is an integer selected from 0, 1 and 2,
with the proviso that the sum of x and y is 2.

Embodiments of the preceding method include the method as just described incorporating one or more of the following features:
(1a) R¹ in the compound of Formula (I) is selected from —CO₂H and -tetrazolyl.
(1b) R¹ in the compound of Formula (I) is —CO₂H.
(2a) R³ in the compound of Formula (I) is selected from the group consisting of phenyl and thienyl, which may be unsubstituted or substituted with 1-5 substituents where the substituents are independently selected from:
   (a) halo,
   (b) trifluoromethyl,
   (c) hydroxy
   (d) C₁₋₃ alkyl, and
   (e) —O—C₁₋₃ alkyl.
(2b) R³ in the compound of Formula (I) is selected from the group consisting of phenyl, which may be unsubstituted or substituted with 1-5 substituents where the substituents are independently selected from (a) fluoro, and (b) chloro; and unsubstituted thienyl.
(2c) R³ in the compound of Formula (I) is unsubstituted phenyl, (3-fluoro)phenyl or 3-thienyl.
(3) R⁴ in the compound of Formula (I) is hydrogen.
(4a) R⁵ in the compound of Formula (I) is selected from hydrogen, C₁₋₆ alkyl, C₃₋₈ cycloalkyl, C₁₋₆ alkyl-C₃₋₈ cycloalkyl, and phenyl.
(4b) R⁵ in the compound of Formula (I) is selected from hydrogen, methyl, n-propyl, isopropyl, n-butyl, t-butyl, isobutyl, sec-butyl, cyclohexyl, —CH₂-cyclopropyl, —CH₂-cyclobutyl, and phenyl.
(4c) R⁵ in the compound of Formula (I) is selected from isopropyl, isobutyl, sec-butyl, and cyclohexyl.
(5a) R⁶ in the compound of Formula (I) is selected from hydrogen, C₁₋₆ alkyl, C₃₋₈ cycloalkyl, C₁₋₆ alkyl-C₃₋₈ cycloalkyl, and phenyl.
(5b) R⁶ in the compound of Formula (I) is selected from hydrogen, methyl, n-butyl, t-butyl, isobutyl, sec-butyl, —CH₂-cyclopropyl, —CH₂-cyclobutyl, and cyclohexyl.
(5c) R⁶ in the compound of Formula (I) is selected from hydrogen, methyl, —CH₂-cyclopropyl, —CH₂-cyclobutyl, and cyclohexyl.
(6a) R⁷ in the compound of Formula (I) is hydrogen, fluoro, hydroxy or C₁₋₆ alkyl.
(6b) R⁷ in the compound of Formula (I) is hydrogen.
(7a) X in the compound of Formula (I) is: —(C₀₋₄ alkyl)-Y—(C₀₋₄ alkyl)-, where the alkyl is unsubstituted or substituted with 1-4 substituents where the substituents are independently selected from:
   (a) halo,
   (b) hydroxy,
   (c) —O—C₁₋₃ alkyl, and
   (d) trifluoromethyl,
      and where Y is selected from:
a single bond, —O—, —SO₂—, —NR¹⁰—, —S—, and —SO—,
   and where R¹⁰ is independently selected from: hydrogen, C₁₋₆ alkyl, C₂₋₆ alkenyl, C₂₋₆ alkynyl, benzyl, phenyl, and C₁₋₆ alkyl-C₃₋₆ cycloalkyl, which is unsubstituted or substituted with 1-3 substituents where the substituents are independently selected from: halo, C₁₋₃ alkyl, C₁₋₃ alkoxy and trifluoromethyl.
(7b) X in the compound of Formula (1) is: —(C₀₋₂ alkyl)-Y—(C₀₋₂ alkyl)-, where the alkyl is unsubstituted or substituted with 1-4 substituents where the substituents are independently selected from:
   (a) halo,
   (b) hydroxy,
   (c) —O—C₁₋₃ alkyl, and
   (d) trifluoromethyl,
      and where Y is selected from:
a single bond, —O—, —SO₂—, —NR¹⁰—, —S—, and —SO—,
   where R¹⁰ is independently selected from: hydrogen, C₁₋₆ alkyl, C₂₋₆ alkenyl, C₂₋₆ alkynyl, benzyl, phenyl, and C₁₋₆ alkyl-C₃₋₆ cycloalkyl,
which is unsubstituted or substituted with 1-3 substituents where the substituents are independently selected from: halo, C₁₋₃ alkyl,
C₁₋₃ alkoxy and trifluoromethyl.
(7c) X in the compound of Formula (I) is selected from —(C₀₋₂ alkyl)-Y—(C₀₋₂ alkyl)-, where the alkyl is unsubstituted or substituted with fluoro,
   and where Y is selected from:
a single bond, —SO₂—, —SO—, and —NR¹⁰—,
   where R¹⁰ is independently selected from: hydrogen, C₁₋₃ alkyl, C₂₋₃ alkenyl, and C₂₋₃ alkynyl.
(7d) X in the compound of Formula (I) is selected from:
   (1) a single bond,
   (2) —CH₂CH₂—,
   (3) —CH₂CH₂CH₂—,
   (4) —CH₂CH₂—CF₂—,
   (5) —CH₂CH₂—SO₂—, and
   (6) —CH₂CH₂—SO—.
(8a) R⁸ in the compound of Formula (I) is selected from: phenyl, naphthyl, cyclohexyl, benzoimidazolyl, benzofurazanyl, imidazopyridyl, imidazolyl, isoxazolyl, oxazolyl, pyrazinyl, pyridazinyl, pyridyl, pyrimidyl, thiazolyl, tetrazolopyridyl, and pyrazolyl;
   which is unsubstituted or substituted with 1-7 substituents where the substituents are independently selected from:
      (a) halo,
      (b) cyano,
      (c) hydroxy,
      (d) C₁₋₆ alkyl, which is unsubstituted or substituted with 1-5 of R¹³ where R¹³ is independently selected from: halo, cyano, hydroxy, C₁₋₆ alkoxy, —CO₂H, —CO₂(C₁₋₆ alkyl), trifluoromethyl, and —NR⁹R¹⁰, wherein R⁹ and R¹⁰ are independently selected from: hydrogen, C₁₋₆ alkyl, C₅₋₆ cycloalkyl, benzyl or phenyl, which is unsubstituted or substituted with 1-3 substituents where the substituents are independently selected from: halo, C₁₋₃ alkyl, C₁₋₃ alkoxy and trifluoromethyl;
      (e) —O—C₁₋₆ alkyl, which is unsubstituted or substituted with 1-5 of R¹³,
      (f) —CF₃,
      (g) —CHF₂,
      (h) —CH₂F,
      (i) —NO₂,
      (j) C₀₋₆ alkyl-phenyl or C₀₋₆ alkyl-heterocycle, which is unsubstituted or substituted with 1-7 substituents where the substituents are independently selected from:
         (i) halo,
         (ii) hydroxy,
         (iii) C₁₋₆ alkyl,
         (iv) —O—C₁₋₆ alkyl,
         (v) —CF₃,
         (vi) —OCF₃,
         (vii) —NO₂,
         (viii) —CN,
         (ix) —SO₂—C₁₋₆ alkyl,
         (x) —CO₂R⁹,
         (xi) —NR⁹R¹⁰,
         (xii) —CONR⁹R¹⁰,
         (xiii) —SO₂—NR⁹R¹⁰, and
         (xiv) —NR⁹—SO₂—R¹⁰;
      (k) —CO₂R⁹,
      (l) tetrazolyl,
      (m) —NR⁹R¹⁰,
      (n) —NR⁹—COR¹⁰,
      (o) —NR⁹—CO₂R¹⁰,
      (p) —CO—NR⁹R¹⁰,
      (q) —OCO—NR⁹R¹⁰,
      (r) —NR⁹CO—NR⁹R¹⁰,
      (s) —S(O)ₘ—R⁹, wherein m is an integer selected from 0, 1 and 2,
      (t) —S(O)₂—NR⁹R¹⁰,
      (u) —NR⁹S(O)₂—R¹⁰, and
      (v) —NR⁹S(O)₂—NR⁹R¹⁰.
(8b) R⁸ in the compound of Formula (I) is selected from phenyl, imidazopyridyl, imidazolyl, oxazolyl, pyrazolyl, pyridyl, and thiazolyl;
   which is unsubstituted or substituted with 1-5 substituents where the substituents are independently selected from:
   (a) halo,
   (b) cyano,
   (c) —NO₂,
   (d) —CF₃,
   (e) —CHF₂,
   (f) —CH₂F,
   (h) C₁₋₆ alkyl,
   (i) C₁₋₃ alkyl-phenyl or C₁₋₃ alkyl-pyridyl, which is unsubstituted or substituted with 1-4 substituents where the substituents are independently selected from:
      (i) halo,
      (ii) C₁₋₆ alkyl,
      (iii) —O—C₁₋₆ alkyl,
      (iv) —CF₃,
      (vi) —OCF₃,
      (vii) —CN, and
   (j) —O—C₁₋₆ alkyl.
(8c) R⁸ in the compound of Formula (I) is selected from imidazolyl, oxazolyl, pyrazolyl, and thiazolyl; which is unsubstituted or substituted with 1-3 substituents where the substituents are independently selected from:
   (a) fluoro,
   (b) cyano,
   (c) C₁₋₃ alkyl,
   (d) —CH₂-phenyl, which is unsubstituted or substituted with 1-4 substituents where the substituents are independently selected from:
      (i) fluoro,
      (ii) chloro,
      (iii) —O—CH₃,
      (iv) —CF₃,
      (v) —CN, and
(e) —CF₃.
(8d) R⁸ in the compound of Formula (I) is selected from 5-(3-benzyl)pyrazolyl, 5-(1-methyl-3-benzyl)pyrazolyl, 5-(1-ethyl-3-benzyl)pyrazolyl, 5-(2-benzyl)thiazolyl, 5-(2-benzyl-4-methyl)thiazolyl, and 5-(2-benzyl-4-ethyl)thiazolyl).
(9) n in the compound of Formula (I) is an integer which is 1.
(10) In the compound of Formula (I), x is an integer which is 1 and y is an integer which is 1.
(11) The subject is a warm-blooded vertebrate.
(12) The subject is a primate.
(13) The subject is a human.
(14) The subject is other than a graft transplant patient.
(15) The subject is a cardiac surgery patient.
(16) The stress response comprises hyperthermia.
(17) The stress response comprises a response to surgery (e.g., surgical hyperthermia).
(18) The stress response comprises hypothermia (e.g., surgical hypothermia).

The present invention also includes a method of treating or preventing stress response in a subject in need thereof, which comprises administering to the subject a compound of Formula (I), or a pharmaceutically acceptable salt or an individual diastereomer thereof, in an amount effective to inhibit endogenous production of one or more pro-inflammatory cytokines selected from the group consisting of IL1, IL6, and TNF (e.g., one or more cytokines selected from the group consisting of IL1 and IL6). Embodiments of this method include the method as just described incorporating one or more of the features (1) to (18) as set forth above for the preceding method directed to Compound I.

The present invention further includes a method of treating or preventing stress response which comprises administering to a subject in need thereof a therapeutically effective amount of a compound of Formula (II), or a pharmaceutically acceptable salt thereof: 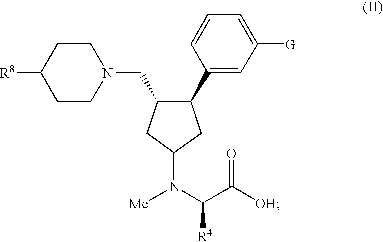
wherein
R⁴is 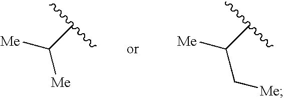
R⁸ is selected from the group consisting of 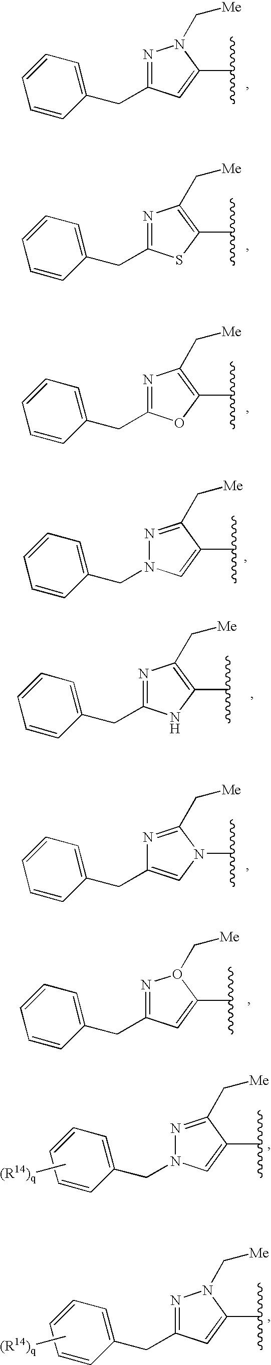 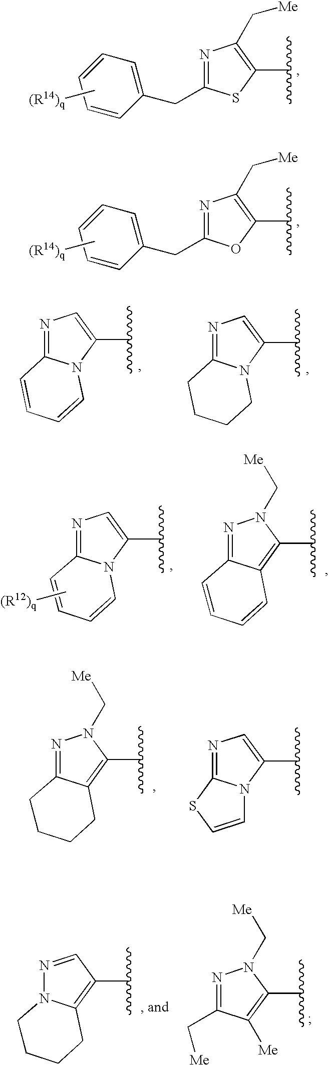
R¹² and R¹⁴ are each independently selected from the group consisting of F, Cl, CF₃, OCH₃, OCH₂CH₃, OCF₃, O-cyclobutyl, CN, O-cyclopropyl, CH₃, CH₂CH₃, CH(CH₃)₂, C(CH₃)₃, and SO₂CH₃;
G is hydrogen or fluoro; and
q is an integer equal to 1 or 2.

Embodiments of the preceding method include the method as just described, except that the stress response comprises hyperthermia, or the stress response comprises a response to surgery, or the stress response comprises hypothermia. Aspects of the method as first described and of the immediately preceding embodiments include the methods in which the subject is a warm-blooded vertebrate, or is a primate, or is a human, or is other than a graft transplant patient, or is a cardiac surgery patient.

The present invention also includes a method of treating or preventing stress response in a subject in need thereof, which comprises administering to the subject a compound of Formula (II), or a pharmaceutically acceptable salt thereof, in an amount effective to inhibit endogenous production of one or more pro-inflammatory cytokines selected from the group consisting of IL1, IL6, and TNF (e.g., one or more cytokines selected from the group consisting of IL1 and IL6). Embodiments of this method include the method as just described incorporating one or more of the embodiments or aspects as set forth in the preceding paragraph directed to related methods involving Compound II.

The present invention also includes a method of treating stress response which comprises administering to a subject in need of such treatment a therapeutically effective amount of Compound A: 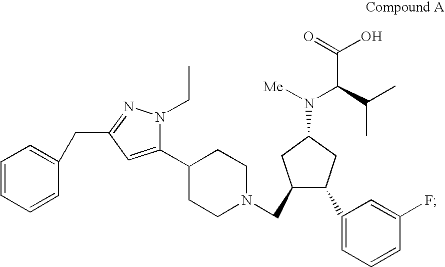
or a pharmaceutically acceptable salt thereof. Embodiments of this method include the method as just described, except that the stress response comprises hyperthermia, or the stress response comprises a response to surgery, or the stress response comprises hypothermia. Aspects of the method as first described and of the immediately preceding embodiments include the methods in which the subject is a warm-blooded vertebrate, or is a primate, or is a human, or is other than a graft transplant patient, or is a cardiac surgery patient.

The present invention also includes a method of treating or preventing stress response in a subject in need thereof, which comprises administering to the subject Compound A, or a pharmaceutically acceptable salt thereof, in an amount effective to inhibit endogenous production of one or more pro-inflammatory cytokines selected from the group consisting of IL1, IL6, and TNF (e.g., one or more cytokines selected from the group consisting of IL1 and IL6). Embodiments of this method include the method as just described incorporating one or more of the embodiments or aspects as set forth in the preceding paragraph directed to related methods involving Compound A.

Compounds of Formula (I), compounds of Formula (II), and Compound A can be prepared as described in U.S. Pat. No. 6,358,979, which is based on U.S. Ser. No. 09/590,750, filed Jun. 8, 2000; and in WO 00/76972, the disclosures of which are herein incorporated by reference in their entireties. These compounds have exhibited activity in binding to the CCR5 receptor, as described in U.S. Pat. No. 6,358,979 and WO 00/76972.

As indicated above, small molecule organic chemokine receptor CCR5 modulators suitable for use in the present invention can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. When the compounds of the invention carry an acidic moiety, suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

Other small molecule organic chemokine receptor CCR5 modulators (especially CCR5 antagonists) suitable for use in the present invention include, for example, those described in U.S. Pat. No. 6,013,644, U.S. Pat. No. 5,962,462, U.S. Pat. No. 5,919,776, U.S. Pat. No. 6,124,319, U.S. Pat. No. 6,136,827, U.S. Pat. No. 6,166,037, U.S. Pat. No. 6,140,349, U.S. Pat. No. 6,265,434, U.S. Pat. No. 6,248,755, WO 00/59498, WO 00/59497, WO 99/76512, WO 00/76511, WO 00/76973, WO 00/76513, and WO 00/76514.

Polypeptides are also suitable for use as chemokine modulators in the present invention. A polypeptide (peptide) modulator interacts with the chemokine receptor CCR5 and can correspond in sequence to a natural ligand. As used herein, the term “polypeptide” refers to a linear or cyclic compound comprising from about 2 to no more than about 100 amino acid residues, wherein the amino group of one amino acid is linked to the carboxyl group of another amino acid by a peptide bond. In one embodiment, polypeptides containing from about 2 to about about 60 residues are employed in the present invention. In another embodiment, polypeptides containing from about 2 to about 30 residues are employed. It is understood that a suitable polypeptide need not be identical to the amino acid residue sequence of a natural ligand, so long as it includes required binding sequences and is able to function as a chemokine receptor CCR5 modulator, especially a CCR5 antagonist.

A suitable polypeptide includes any analog, fragment or chemical derivative of a polypeptide that is a chemokine receptor CCR5 modulator. Such a polypeptide can be subject to various changes, substitutions, insertions, and deletions where such changes provide for certain advantages in its use (e.g., improvement in the potency of the polypeptide or conversion of the polypeptide from an agonist to an antagonist of the CCR5 receptor). A modulator polypeptide suitable for use in the present invention can correspond to, rather than be identical to, the sequence of a natural ligand where one or more changes are made in the sequence and it retains the ability to function as a chemokine receptor CCR5 modulator. Thus, a suitable polypeptide can be in any of a variety of forms of peptide derivatives, that include amides, conjugates with proteins, cyclized peptides, polymerized peptides, analogs, fragments, chemically modified peptides, and the like, provided it is a modulator of chemokine receptor CCR5 activity.

The “analog” of a polypeptide refers to any polypeptide having an amino acid residue sequence substantially identical to a sequence of a natural ligand of a chemokine receptor CCR5 in which one or more residues have been conservatively substituted with a functionally similar residue and which displays the requisite chemokine receptor modulator activity. Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another. The phrase “conservative substitution” also includes the use of a chemically derivatized residue in place of a non-derivatized residue provided that such polypeptide displays the requisite inhibition activity. The phrase “chemical derivatized polypeptide” refers to a subject polypeptide having one or more residues chemically derivatized by reaction of a functional side group. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-imbenzylhistidine. Also included as chemical derivatives are those peptides that contain one or more naturally occurring amino acid derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

Additional residues can also be added at either terminus of a polypeptide for the purpose of providing a “linker” by which the polypeptides of the present invention can be conveniently affixed to a label or solid matrix, or carrier. Labels, solid matrices and carriers that can be used with the polypeptides of the present invention are described hereinbelow. Amino acid residue linkers are usually at least one residue and can be 40 or more residues, more often 1 to 10 residues, but do not form chemokine receptor ligand epitopes. Typical amino acid residues used for linking are tyrosine, cysteine, lysine, glutamic and aspartic acid, or the like. In addition, a subject polypeptide can differ, unless otherwise specified, from the natural sequence of a ligand by the sequence being modified by terminal-NH2 acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half life of the polypeptides in solutions, particularly biological fluids where proteases can be present. In this regard, polypeptide cyclization is also a useful terminal modification, and is particularly preferred also because of the stable structures formed by cyclization.

Any polypeptide suitable for use in the present invention can be employed in the form of a pharmaceutically acceptable salt. Suitable acids which are capable of forming salts with the peptides include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. Suitable bases capable of forming salts with the peptides of the present invention include inorganic bases such as sodium hydroxide, ammonium hydroxide, potassium hydroxide and the like; and organic bases such as mono-di- and tri-alkyl and aryl amines (e.g. triethylamine, diisopropyl amine, methyl amine, dimethyl amine and the like), and optionally substituted ethanolamines (e.g. ethanolamine, diethanolamine and the like).

A polypeptide suitable for use in the present invention can be synthesized by techniques known to those skilled in the polypeptide art, including recombinant DNA techniques. Synthetic chemistry techniques, such as a solid-phase Merrifield-type synthesis, are preferred for reasons of purity, antigenic specificity, freedom from undesired side products, ease of production and the like. Suitable techniques for preparing polypeptides include those described in Steward et al., “Solid Phase Peptide Synthesis”, W. H. Freeman Co., San Francisco, 1969; Bodanszky et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol. 1969, 32: 221-96; Fields et al., Int. J. Peptide Protein Res. 1990, 35: 161-214; U.S. Pat. No. 4,244,946 for solid phase peptide synthesis; and Schroder et al., “The Peptides”, Vol. 1, Academic Press (New York), 1965 for classical solution synthesis; each of which is incorporated herein by reference in its entirety. Appropriate protective groups usable in such syntheses are described in the above texts and in J. F. W. McOmie, “Protective Groups in Organic Chemistry”, Plenum Press, New York, 1973, which is incorporated herein by reference in its entirety, and in T. W. Greene and P. G. M. Wuts, “Protective Groups in Organic Synthesis”, 2^{nd} edition, John Wiley & Sons, New York, 1991, which is incorporated herein by reference in its entirety.

In general, the solid-phase synthesis methods comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

In a representative solid phase synthesis, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted under conditions suitable for forming the amide linkage with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to afford the final linear polypeptide.

Linear polypeptides, such as a linear peptide prepared by a solid phase synthesis as just described, can be reacted to form their corresponding cyclic peptides. An exemplary method for cyclizing peptides is described on pages 393-394 of Zimmer et al., “Peptides 1992”, ESCOM Science Publishers, B. V., 1993, which is herein incorporated by reference in its entirety. Typically, tert-butoxycarbonyl protected peptide methyl ester is dissolved in methanol, sodium hydroxide solution is added, and the admixture is reacted at 20° C. to hydrolytically remove the methyl ester protecting group. After evaporating the solvent, the tert-butoxycarbonyl protected peptide is extracted with ethyl acetate from acidified aqueous solvent. The tert-butoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

Antibodies are also suitable for use as chemokine receptor CCR5 modulators in the present invention, wherein the antibodies, including monoclonal antibodies, immunoreact with a chemokine receptor CCR5 and/or bind the chemokine receptor to modulate receptor activity. The term “antibody”, or a variant thereof (e.g., “antibody molecule”), refers to a population of immunoglobulin molecules and/or immunologically active portions of immunoglobulin molecules; i.e., molecules that contain an antibody combining site or paratope. An “antibody combining site” is that structural portion of an antibody molecule comprised of heavy and light chain variable and hypervariable regions that specifically binds antigen.

Exemplary antibodies suitable for use in the present invention are intact immunoglobulin molecules, substantially intact immunoglobulin molecules (i.e., immunoglobulins having changes in sequence that do not affect its ability to fix complement or to interact with Fc receptors), single chain immunoglobulins or antibodies, those portions of an immunoglobulin molecule that contain the paratope, including those portions known in the art as Fab, Fab′, F(ab′)2 and F(v), and also referred to as antibody fragments. The phrase “monoclonal antibody”, or a variant thereof, refers to a population of antibody molecules that contain only one species of antibody combining site capable of immunoreacting with a particular epitope. A monoclonal antibody thus typically displays a single binding affinity for any epitope with which it immunoreacts. A monoclonal antibody can therefore contain an antibody molecule having a plurality of antibody combining sites, each immunospecific for a different epitope, e.g., a bispecific monoclonal antibody.

A monoclonal antibody is typically composed of antibodies produced by clones of a single cell called a hybridoma that secretes (produces) only one kind of antibody molecule. The hybridoma cell is formed by fusing an antibody-producing cell and a myeloma or other self-perpetuating cell line. The preparation of such antibodies was first described by Kohler and Milstein, Nature 1975, 256: 495-497, which description is incorporated by reference in its entirety. Additional methods are described by Zola, “Monoclonal Antibodies: a Manual of Techniques”, CRC Press, Inc., 1987. The hybridoma supernates so prepared can be screened for the presence of antibody molecules that immunoreact with a chemokine receptor and modulate its biological function.

Briefly, to form the hybridoma from which the monoclonal antibody composition is produced, a myeloma or other self-perpetuating cell line is fused with lymphocytes obtained from the spleen of a mammal hyperimmunized with a source of a chemokine receptor, as described by Cheresh et al., J. Biol. Chem. 1987, 262: 17703-17711, herein incorporated by reference in its entirety. It is preferred that the myeloma cell line used to prepare a hybridoma be from the same species as the lymphocytes. Typically, a mouse of the strain 129 G1X+ is the preferred mammal. Suitable mouse myelomas include the hypoxanthine-aminopterin-thymidine-sensitive (HAT) cell lines P3X63-Ag8.653, and Sp2/0-Ag14 that are available from the ATCC, Manassas, Va., under the designations CRL 1580 and CRL 1581, respectively. Splenocytes are typically fused with myeloma cells using polyethylene glycol (PEG) 1500. Fused hybrids are selected by their sensitivity to HAT. Hybridomas producing a monoclonal antibody of the present invention can be identified using the enzyme linked immunosorbent assay.

A suitable monoclonal antibody can also be produced by initiating a monoclonal hybridoma culture comprising a nutrient medium containing a hybridoma that secretes antibody molecules of the appropriate specificity. The culture is maintained under conditions and for a time period sufficient for the hybridoma to secrete the antibody molecules into the medium. The antibody-containing medium is then collected. The antibody molecules can then be further isolated by well known techniques. Media useful for the preparation of these compositions are both well known in the art and commercially available and include synthetic culture media, inbred mice and the like. An exemplary synthetic medium is Dulbecco's minimal essential medium (DMEM—Dulbecco et al., Virol. 1959, 8: 396) supplemented with 4.5 gm/1 glucose, 20 mM glutamine, and 20% fetal calf serum. An exemplary inbred mouse strain is the Balb/C.

Other methods of producing a monoclonal antibody, a hybridoma cell, or a hybridoma cell culture include, for example, the method of isolating monoclonal antibodies from an immunological repertoire as described by Sastry, et al., Proc Natl. Acad. Sci. USA 1989, 86: 5728-5732; and Huse et al., Science 1989, 246: 1275-1281, each of which is herein incorporated by reference in its entirety. Also suitable for use in the present invention are monoclonal antibodies produced from cultures containing a hybridoma cell.

It is also possible to determine, without undue experimentation, if a monoclonal antibody has the same (i.e., equivalent) specificity (immunoreaction characteristics) as a monoclonal antibody suitable for use in the present invention by ascertaining whether the former prevents the latter from binding to a preselected target molecule. If the monoclonal antibody being tested competes with the monoclonal antibody of the invention, as shown by a decrease in binding by the monoclonal antibody of the invention in standard competition assays for binding to the target molecule when present in the solid phase, then it is likely that the two monoclonal antibodies bind to the same, or a closely related, epitope.

Still another way to determine whether a monoclonal antibody has the specificity of a monoclonal antibody of the invention is to pre-incubate the monoclonal antibody of the invention with the target molecule with which it is normally reactive, and then add the monoclonal antibody being tested to determine if the monoclonal antibody being tested is inhibited in its ability to bind the target molecule. If the monoclonal antibody being tested is inhibited then, in all likelihood, it has the same, or functionally equivalent, epitopic specificity as the monoclonal antibody of the invention.

An additional way to determine whether a monoclonal antibody has the specificity of a monoclonal antibody of the invention is to determine the amino acid residue sequence of the complementarity determining regions (CDRs) of the antibodies in question. Antibody molecules having identical, or functionally equivalent, amino acid residue sequences in their CDRs have the same binding specificity. Methods for sequencing polypeptides are well known in the art.

The immunospecificity of an antibody, its target molecule binding capacity, and the attendant affinity the antibody exhibits for the epitope, are defined by the epitope with which the antibody immunoreacts. The epitope specificity is defined at least in part by the amino acid residue sequence of the variable region of the heavy chain of the immunoglobulin that comprises the antibody, and in part by the light chain variable region amino acid residue sequence. Use of the terms “having the binding specificity of” or “having the binding preference of” indicates that equivalent monoclonal antibodies exhibit the same or similar immunoreaction (binding) characteristics and compete for binding to a preselected target molecule. Humanized monoclonal antibodies offer particular advantages over murine monoclonal antibodies, particularly insofar as they can be used therapeutically in humans. Specifically, human antibodies are not cleared from the circulation as rapidly as “foreign” antigens, and do not activate the immune system in the same manner as foreign antigens and foreign antibodies. Methods of preparing “humanized” antibodies are generally well known in the art, and can readily be applied to the antibodies of the present invention. Thus, the invention provides, in one embodiment, a monoclonal antibody of the present invention that is humanized by grafting to introduce components of the human immune system without substantially interfering with the ability of the antibody to bind antigen. Humanized antibodies can also be produced using animals engineering to produce humanized antibodies, such as those available from Medarex of Annandale, N.J. (mice) and Abgenix, Inc., of Fremont, Calif. (mice). The use of a molecular cloning approach to generate antibodies, particularly monoclonal antibodies, and more particularly single chain monoclonal antibodies, is also provided.

The production of single chain antibodies has been described in the art, as for example in U.S. Pat. No. 5,260,203, the contents of which are herein incorporated by reference. For this, combinatorial immunoglobulin phagemid or phage-displayed libraries are prepared from RNA isolated from the spleen of the immunized animal, and phagemids expressing appropriate antibodies are selected by panning on endothelial tissue. This approach can also be used to prepared humanized antibodies. The advantages of this approach over conventional hybridoma techniques are that approximately 10⁴ times as many antibodies can be produced and screened in a single round, and that new specificities are generated by H and L chain combination in a single chain, which further increases the chance of finding appropriate antibodies. Thus, an antibody suitable for use in the present invention, or a “derivative” of an antibody of the present invention pertains to a single polypeptide chain binding molecule which has binding specificity and affinity substantially similar to the binding specificity and affinity of the light and heavy chain aggregate variable region of an antibody described herein.

“Fv” is the minimum antibody fragment that contains a complete antigen-recognition and -binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. For a review of scFv see Pluckthun, in The Pharmacology of Monoclonal Antibodies, vol. 113, Rosenburg and Moore eds., Springer-Verlag, New York, pp. 269-315 (1994).

The present invention also includes the use of a CCR5 antagonist for treating or preventing stress response in a subject in need thereof. The present invention further includes the use of a CCR5 antagonist in the manufacture of a medicament for treating or preventing stress response in a subject in need thereof. Embodiments of these uses are the uses as just described incorporating one or more of the embodiments, aspects and features of any one or more of the previously described treatment and prevention methods of the invention. Thus, embodiments of the above-described uses include uses in which the CCR5 antagonist is a compound of Formula (I), or is a compound of Formula (II), or is Compound A; and/or the subject is a warm-blooded vertebrate, or is a primate, or is a human; and/or the subject is other than a graft transplant patient, or is a cardiac surgery patient; and/or the stress response comprises hyperthermia (or hypothermia); and/or the use is inhibiting hyperthermia (or hypothermia).

The present invention also includes a method for treating or preventing stress response in a subject in need thereof which comprises administering to the subject a therapeutically effective amount of a CCR5 antagonist and a therapeutically effective amount of an immunosuppressive agent. The CCR5 antagonist can be administered before, concurrently with, or after administration of the immunosuprressive agent. The term “immunosuppressive agent” refers to compounds which can inhibit an immune response. In one embodiment, the immunosuppressive agent is selected from the group consisting of calcineurin inhibitors (e.g., cyclosporin A, FK506), IL2 signal transduction inhibitors (e.g., rapamycin), glucocorticoids (e.g., prednisone, dexamethasone, methylprednisolone, prednisolone), nucleic acid synthesis inhibitors (e.g., azathioprine, mercaptopurine, mycophenolic acid), antibodies to lymphocytes or antigen-binding fragments thereof (e.g., OKT3, anti-EL2 receptor, anti-CD52), and lymphocyte sequestrants (e.g., FTY720). In another embodiment, the immunosuppressive agent is a calcineurin inhibitor. In an aspect of this embodiment, the calcineurin inhibitor is cyclosporin A. In still another embodiment, the immunosuppressive agent is a lymphocyte sequestrant. Additional embodiments of this method is the method as first described or as described in one of the foregoing embodiments incorporating one or more of the embodiments, aspects and features of any one or more of the previously described treatment and prevention methods of the invention. Thus, for example, one embodiment of this method is the method in which the subject is other than a graft transplant patient.

The present invention also includes a method of treating or preventing a disorder characterized by the activity of at least one pro-inflammatory cytokine selected from the group consisting of IL1, IL6 and TNF (e.g., at least one cytokine selected from IL1 and IL6), in a mammal (e.g., a primate, especially a human) in need of such treatment or prevention, which comprises administering to the mammal a CCR5 modulator in an amount effective to inhibit endogenous production of the cytokine. In one embodiment of this method, the CCR5 modulator comprises a CCR5 antagonist. In an aspect of this embodiment, the CCR5 antagonist comprises a small molecule organic compound, a polypeptide, or an antibody. Features of this aspect include the method wherein the CCR5 modulator is a compound of Formula (I) or a pharmaceutically acceptable salt or individual diastereomer thereof, a compound of Formula (II) or a pharmaceutically acceptable salt thereof, or Compound A or a pharmaceutically acceptable salt thereof, each as heretofore defined and described. In another embodiment, the disorder being treated or prevented is selected from the group consisting of post-surgical inflammatory response, sepsis, septic shock, and acute respiratory distress syndrome (ARDS). Aspects and features of this embodiment include aspects and features described above for the previous embodiment.

The present invention also includes a method of treating a post-trauma inflammatory response in a subject undergoing or having undergone a multiple trauma associated with a high risk of sepsis or ARDS, which comprises administering to the subject a therapeutically effective amount of a CCR5 antagonist. Exemplary multiple traumas pelvic or multiple long bone fracture, massive blood loss, multiple unit blood transfusion, prolonged hypotension/shock, and pulmonary contusion. In one embodiment of this method, the CCR5 antagonist comprises a small molecule organic compound, a polypeptide, or an antibody. Aspects of this embodiment include the method wherein the CCR5 antagonist is a compound of Formula (I) or a pharmaceutically acceptable salt or individual diastereomer thereof, a compound of Formula (II) or a pharmaceutically acceptable salt thereof, or Compound A or a pharmaceutically acceptable salt thereof, each as heretofore defined and described.

The term “post-surgical inflammatory response” as used herein refers to any disease, symptom, or pathological condition resulting from an excessive or unregulated inflammatory response following surgery that can be attributed to the induction of at least one of the pro-inflammatory cytokines IL1, IL6 and TNF.

The term “sepsis” (also known in the art as systemic inflammatory response syndrome (SIRS)) refers to a syndrome in which immune mediators, such as pro-inflammatory cytokines, produced or released in response to, for example, microbial invasion (e.g., by gram-negative bacteria with concomitant endotoxin infusion), injury (e.g., multiple long bone fracture) or other insults (e.g., burns), induce an acute state of inflammation which leads to abnormal homeostasis, organ damage and eventually to lethal shock. Individuals with sepsis typically exhibit fever, tachycardia, tachypnea, leukocytosis, and a localized site of infection. Microbiologic cultures from blood or the infection site are frequently, but not always, positive. “Septic shock” can occur subsequent to sepsis and refers to the condition in which pathogenic microorganisms, typically gram-negative bacteria, or their toxins are present in the blood or in other tissues during infection. Its symptoms typically include a drop in blood pressure, fever, diarrhea, widespread blood clotting in various organs, and ultimately organ failure. “Acute respiratory distress syndrome” (ARDS) refers to lung-failure related pathophysiology that is typically the first of the multiple organ failures that characterize the terminal phase of sepsis and septic shock.

The present invention also includes a method of inhibiting endogenous production of at least one pro-inflammatory cytokine selected from the group consisting of IL1, IL6, and TNF (e.g., at least one cytokine selected from IL1 and IL6), which comprises administering to a mammal in need of such inhibition a CCR5 modulator in an amount effective to inhibit production of the cytokine. In one embodiment of this method, the CCR5 modulator comprises a CCR5 antagonist. In an aspect of this embodiment, the CCR5 antagonist comprises a small molecule organic compound, a polypeptide, or an antibody. Features of this aspect include the method wherein the CCR5 modulator is a compound of Formula (I) or a pharmaceutically acceptable salt or individual diastereomer thereof, a compound of Formula (II) or a pharmaceutically acceptable salt thereof, or Compound A or a pharmaceutically acceptable salt thereof, each as heretofore defined and described.

The present invention further includes a method for monitoring the effectiveness of treatment of a subject (typically a mammal, preferably a primate, more preferably a human) suffering from an acute inflammatory response, said treatment comprising administration of a CCR5 modulator, wherein the method comprises:
(A) obtaining a pre-administration sample (e.g., a serum or tissue sample) from the subject prior to administration of the CCR5 modulator and determining the level of expression or activity of a pro-inflammatory cytokine selected from the group consisting of IL1, IL6 and TNF (e.g., a cytokine selected from IL1 and IL6) in the pre-administration sample;
(B) obtaining a post-administration sample from the subject subsequent to administration of the CCR5 modulator and determining the level of expression or activity of the pro-inflammatory cytokine; and
(C) comparing the level of cytokine expression or activity of the post-administration sample with the level of cytokine expression or activity of the pre-administration sample.

An embodiment of this method is the method as just described, which further comprises:
(D) adjusting the administration of the CCR5 modulator to increase or decrease the level of cytokine expression or activity; and
(E) repeating steps (A), (B), and (C).

In an aspect of this method and its foregoing embodiment, the CCR5 modulator comprises a CCR5 antagonist. In another aspect, the CCR5 modulator comprises a CCR5 antagonist which comprises a small molecule organic compound, a polypeptide, or an antibody. The CCR5 antagonist can be a compound of Formula (I) or a pharmaceutically acceptable salt or individual diastereomer thereof, a compound of Formula (II) or a pharmaceutically acceptable salt thereof, or Compound A or a pharmaceutically acceptable salt thereof, each as heretofore defined and described.

The level of expression or activity of IL1, IL6, and TNF can be determined from serum, plasma, or whole blood samples in accordance with procedures described in Casey et al., Ann. Intern. Med. 1993, 119(8): 771-778, and in Bolke et al. Shock 2001, 16(5): 334-9. Cytokine levels can also be determined by flow cytometry using, for example, Becton Dickinson's Cytometric Bead Array Technology.

The present invention also includes a method for determining the efficacy of a CCR5 modulator in correcting an abnormal level of a pro-inflammatory cytokine selected from the group consisting of IL1, IL6 and TNF (e.g., a cytokine selected from IL1 and IL6) in a subject in need of such correction, which comprises:
(A) administering an amount of the CCR5 modulator to the subject; and
(B) determining the level of the cytokine in the subject following administration of the CCR5 modulator, wherein a change in the cytokine level toward a normal level is a measure of the efficacy of the modulator.

An embodiment of this method is a method for determining the efficacy of a CCR5 antagonist in reducing an abnormally high level of a pro-inflammatory cytokine selected from the group consisting of IL1, IL6 and TNF (e.g., a cytokine selected from IL1 and IL6) in a subject in need of such reduction, which comprises:
(A) administering an amount of the CCR5 antagonist to the subject; and
(B) determining the level of the cytokine in the subject following administration of the CCR5 antagonist, wherein a reduction in the cytokine level toward a normal level is a measure of the efficacy of the antagonist.

In an aspect of this embodiment, the CCR5 antagonist comprises a small molecule organic compound, a polypeptide, or an antibody. In still another aspect, the CCR5 antagonist can be a compound of Formula (I) or a pharmaceutically acceptable salt or individual diastereomer thereof, a compound of Formula (II) or a pharmaceutically acceptable salt thereof, or Compound A or a pharmaceutically acceptable salt thereof, each as heretofore defined and described.

The term “abnormal level” (which may also be referred to as an “aberrant level”) refers to a level of a pro-inflammatory cytokine (IL1, IL6 or TNF) which is measurably different (whether higher or lower) from the level of the cytokine in the healthy subject. As already noted above, because the normal level of cytokine can vary from one subject to the next, it is preferred to determine the normal cytokine level for the particular subject prior to exposure to an insult (e.g., prior to a planned insult such as surgery). Alternatively, the normal cytokine level can be equated to the average value obtained or known for a group of similarly situated healthy individuals.

The activity of agents (e.g., small molecule organics, polypeptides, antibodies) as chemokine receptor CCR5 modulators can be determined using methods known in the art. More particularly, the activity of an agent as a CCR5 receptor modulator can be determined using a suitable screen (e.g., high through-put assay). For example, an agent can be tested in an extracellular acidification assay, calcium flux assay, ligand binding assay or chemotaxis assay. Suitable assays are described, for example, in Hale et al., Bioorg. & Med. Chem. Letters 2001, 11: 1437-1440; Hesselgesser et al., J. Biol. Chem. 1998, 273 (25): 15687-15692; WO 98/18826 and WO 98/02151, the disclosures of which are incorporated herein by reference. Also suitable for assessing the activity of agents as CCR5 modulators are the assays described in WO 01/78707, the disclosure of which is incorporated herein by reference.

The active agent(s) (i.e., a chemokine receptor CCR5 modulator and optionally one or more additional therapeutic agents) can be administered orally, parenterally (including intravenous, intramuscular, or intrasternal injection, or infusion techniques), by subcutaneous administration (e.g., injection), by inhalation spray, by buccal delivery, by surgical implantation, or rectally, in the form of a unit dosage of a pharmaceutical composition containing a therapeutically effective amount of the modulator (e.g., a small organic molecule) and conventional non-toxic pharmaceutically-acceptable carriers, adjuvants and vehicles. The particular mode of drug administration used in accordance with the methods of the present invention depends on various factors, including but not limited to the severity of the condition to be treated and mechanisms for metabolism or removal of the drug following administration. Oral and parenteral administration are generally preferred, however.

Suitable formulations for injection include aqueous and non-aqueous sterile solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

For oral administration, the compositions can take the form of, for example, tablets or capsules prepared by conventional techniques with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods known in the art. For example, a CCR5 antagonist can be formulated in combination with hydrochlorothiazide, and as a pH stabilized core having an enteric or delayed release coating which protects the CCR5 antagonist until it reaches the colon.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in a conventional manner.

For rectal administration, the agents (e.g., CCR5 antagonist and optionally one or more additional therapeutic agents) can be formulated as suppositories or retention enemas containing conventional suppository bases such as cocoa butter or other glycerides).

The agents can also be formulated as creams or lotions, or transdermal patches.

The amount of the agent (e.g., CCR5 receptor modulator) administered to the subject can be varied so as to administer an amount that is effective to achieve the desired therapeutic response. The selected dosage level and frequency of administration thereof will depend upon a variety of factors including the activity of the composition, choice of formulation, the route of administration, combination with other drugs or treatments, the severity of the condition being treated, and the physical condition (e.g., health, age, sex, weight, diet) and prior medical history of the subject being treated. In one embodiment, a minimal dose is administered, and dose escalated in the absence of dose-limiting toxicity to a minimally effective amount. In another embodiment, the agent is administered in an amount of from about 0.001 to about 1000 mg/kg of body weight of the subject per day in a single dose or in divided doses. A preferred dosage range is from about 0.01 to about 500 mg/kg body weight per day in a single dose or divided doses. In still another embodiment, the agent is administered in a range of from about 0.1 to 100 mg per day to an adult human.

It is understood that when two or more agents are administered, the agents can be administered concurrently or sequentially in either order, with a suitable mode of administration and dosage form selected for each. When the agents are administered at different times, the time interval between administrations is suitably selected such that there is an overlap in the efficacy and activity of each of the agents, whereby an additive or synergistic benefit is provided. The person of ordinary skill in the art can determine the appropriate order of and time interval between administration of the agents and can also determine the appropriate dosage level and form for each agent.

For purposes of prevention, the CCR5 modulator is suitably administered before the insult (when such insult is planned such as elective surgery) or during or after its occurrence but before the stress response manifests itself. In the case of surgical insult, the time for administration is chosen such that the agent will provide efficacy and pharmacological activity at the time the insult is scheduled to occur. The agent is suitably administered within about about 24 hours (e.g., from about 1 to about 12 hours) prior to insult, and is typically administered within about 8 hours (e.g., from about 0.5 to about 4 hours) prior to surgery. The timing will depend upon the pharmacokinetics of the agent, mode of administration, the physical condition of the patient, and other factors related to administration frequency and dosage level described earlier.

Additional guidance regarding formulation and dose can be found in U.S. Pat. No. 5,326,902; U.S. Pat. No. 5,234,933; WO 93/25521; Berkow et al. (1997) The Merck Manual of Medical Information, Home ed. Merck Research Laboratories, Whitehouse Station, N.J.; Goodman et al. (1996) Goodman & Gilman's the Pharmacological Basis of Therapeutics, 9th ed. McGraw-Hill Health Professions Division, New York; Ebadi (1998) CRC Desk Reference of Clinical Pharmacology. CRC Press, Boca Raton, Fla.; Katzung (2001) Basic & Clinical Pharmacology, 8th ed. Lange Medical Books/McGraw-Hill Medical Pub. Division, New York; Remington et al. (1975) Remington's Pharmaceutical Sciences, 15th ed. Mack Pub. Co., Easton, Pa.; and Speight et al. (1997) Avery's Drug Treatment: A Guide to the Properties, Choice, Therapeutic Use and Economic Value of Drugs in Disease Management, 4th ed. Adis International, Auckland/Philadelphia; Duch et al. (1998) Toxicol Lett 100-101:255-263.

The following examples serve only to illustrate the invention and its practice. The examples are not to be construed as limitations on the scope or spirit of the invention.

### EXAMPLE 1

### Fever Suppression after Cardiac Allotransplantation

This example shows fever suppression in a group of monkeys treated with a CCR5 antagonist after undergoing cardiac allotransplantation. Cynomolgus macaques (Macaca fascicularis), weighing 2.4-4.7 kg, and ranging in estimated age from 2.2-5.5 years were selected as organ recipients. Donors were matched to recipients by AB blood type compatibility as tested by New York University, Nelson Institute of Environmental Medicine, Tuxedo, N.Y. MHC class II mismatch was assured by a stimulation index (SI)>3 in paired animals in unidirectional mixed lymphocyte reaction (MLR) using potential donor lymphocytes as stimulators and potential recipient peripheral blood mononuclear cells (PBMCs) as responders. Animals were matched by assigning pairs with maximal MLR response within groups of blood type compatible animals.

Anesthesia: Anesthesia was induced with intramuscular ketamine (10 mg/kg) (Fort Dodge Animal Health, Fort Dodge, Iowa) and glycopyrrolate (0.01 mg/kg IM) (Fort Dodge Animal Health, Fort Dodge, Iowa) and maintained with propofol (i.e., 2,6-diisopropylphenol) (Abbott Laboratories, Chicago, Ill.) to effect a surgical plane of anethesia. In animals with established venous access, anesthesia was induced with propofol.

Donor operation: After systemic heparinization (70 units/kg, Elkins-Sinn Inc., Cherry Hill, N.J.) and collection of donor blood during equivolemic saline infusion, diastolic arrest of the donor heart was induced with University of Wisconsin organ preservation solution (15-20 cc/kg, 4° C.) via the aortic root. The heart was explanted and prepared: The mitral valve was rendered incompetent surgically, to prevent left ventricular distention, and an atrial septal defect was created by excising the fossa ovalis, to allow blood entering the left heart through the thebesian circulation to transit to and be ejected from the right heart. The left atrium was oversewn, and the supra vena cava (SVC) and inferior vena cava (IVC) ligated.

Recipient operation: All recipient animals underwent traditional non-working intraabdominal cardiac allograft transplantation using a single clamp technique, wherein the donor aorta was anastomosed end-to-side to the infrarenal abdominal aorta, and the donor pulmonary artery was anastomosed to the adjacent vena cava. Heparin (70 Units/kg) (a Unit is equivalent to 3 mg/kg of body weight) was administered to the recipient prior to clamp placement. A silicon central venous catheter was introduced via the internal jugular vein, and tunneled to exit between the scapulae. Catheters were attached to swivel connection systems, and animals placed in protective cloth jackets incorporating a swiveling system which protected venous access.

Postoperative analgesia consisted of intramuscular buprenorphine 0.01 mg/kg (Reckitt & Colman Pharm., Richmond, Va.) and ketoprofen 2 mg/kg (Fort Dodge Animal Health, Fort Dodge, Iowa).

Graft function and body temperature were assessed twice daily by monitoring with a fully implantable telemetry device (Data Sciences International, St. Paul, Minn.) (electrocardiogram (EKG), right ventricular pressure and animal body temperature) implanted at the time of transplantation. Confirmatory abdominal ultrasounds were performed at the time of protocol biopsies and whenever an examiner appreciated decreased contractility, if EKG voltage or rate (<120 beats per minute) was decreased, or if developed pulse pressure (delta P) in the graft was decreased. Cardiac biopsies were performed by protocol on postoperative days 4, 7, 14, through a laparotomy incision using a core biopsy needle. Graft failure was defined as loss of palpable graft activity, with ultrasound confirmation of weak or absent myocardial contractility. Failed grafts were explanted promptly and examined histologically.

Blood draws were performed from a peripheral site on the days of surgical intervention to monitor complete blood counts and blood chemistry for eventual drug related toxicity or side effects.

Three animals received Compound A as monotherapy at a dose of 5 mg/kg, two animals received Compound A as monotherapy at a dose of 10 mg/kg, and three animals received an equivalent volume of saline (control). Compound A was administered intravenously twice daily at 12 hour intervals (i.e., b.i.d.) starting at transplant. Two other animals received Compound A at 5 mg/kg b.i.d. combined with a subtherapeutic amount cyclosporin A (CsA), and a further two animals (control) received CsA only. The CsA was dosed by daily intramuscular injection. The dose on the first day was 12.5 mg/kg, then 10 mg/kg daily for 7 days, then 5 mg/kg daily for 7 days, then 2.5 mg/kg/day until graft rejection or animal sacrifice. In one instance, an animal initially treated with Compound A alone (10 mg/kg b.i.d.) was rescued on day 9 with bolus steroids (40 mg/kg on day 1; 20 mg/kg SID on days 2 and 3) and then treated with combined therapy for 43 days. On day 52, CsA treatment was stopped, and the animal remained on a twice daily regimen of Compound A (10 mg/kg). Vigorous graft function persisted to day 83, 30 days after stopping CsA.

Post transplantation, the animals were individually housed in stainless steel metal caging, maintained at 22° C. with 12-hour light/dark cycles. Tap water was available ad libidum, and the monkeys were fed commercial primate chow and fruit.

The febrile response characteristic of the first three days after allograft implantation in control animals and animals treated with conventional immunosuppression (i.e., CsA) was not seen in CCR5 antagonist-treated animals. In the three control animals that received saline infusions, the average temperature was consistently above 38° C. Similarly in the two animals treated with CsA alone, fever was observed in one of the animals. In total, fever was observed in 4 of the 5 animals not receiving Compound A, and was typically recurrent in those animals. In contrast, the average temperature in six of the seven animals treated with Compound A was below 37.5° C., and individual temperature elevations reached 38.5° C. on any occasion in only 3 of the animals. In summary, six of seven monkeys treated with a CCR5 antagonist did not develop a fever (i.e., a temperature greater than 38.5° C.) while recovering from the transplantation procedure, and had an average temperature about one degree lower than the control monkeys receiving no immunosuppressive therapy and one half degree lower than animals in which acute rejection was prevented by CsA therapy. In addition, in contrast to the five control animals, the CCR5 antagonist-treated monkeys were observed not to exhibit malaise and to behave as if they had not had surgery. The treated monkeys also did not exhibit any symptoms of abdominal tenderness that would otherwise have been expected, despite major biochemical perturbations including increased creatinine and bilirubin (as determined by analysis of the serum chemistries) that would normally have been associated with discomfort and malaise. These findings indicate that CCR5 plays a previously unrecognized role in the acute phase inflammatory response.

Double-label immunofluorescence microscopy using antibodies specific for CD3, CD68, CD11b, CCR5 and CXCR3 was used to characterize leukocyte populations infiltrating acutely rejecting (days 4, 6) cynomolgus cardiac allografts. CCR5 and CXCR3 are prominent on monocytes/macrophages and to a lesser extent on T-cells in rejecting cynomolgus heart allografts. Co-localization of CCR5 and CXCR3 to the same cells was common, particularly on the D68 monocyte/macrophage population. Infiltration of CCR5+ cells was inhibited in association with CCR5 blockade. Graft failure was identified by a decrease in heart rate and ST elevation (associated with ischemia) on EKG and a decrease in arterial line pressure. Graft survival was prolonged from 6±0.4 days (n=7) to 8.3±0.6 days (n=3) (p=0.05) (n=3 refers to the animals who received 5 mg/kg Compound A monotherapy) despite pharmacokinetics data for Compound A (i.e. trough concentrations determined by LC-MS/MS following solid phase extraction of plasma using Waters OASIS 96-well extraction plate (30 mg)) that suggested full receptor coverage may not have been achieved at trough drug concentrations; i.e., the levels of Compound A fell below the target range at trough of 100 nM, a concentration which corresponded to about 90% receptor occupancy. (Note: n=7 refers to the data for the 3 control (i.e., saline) monkeys in this experiment plus data for 4 animals from a historic database that were scored as controls in which cyno cardiac allografts were rejected in the absense of immunosupression in a manner identical to the three controls in this experiment.)

Administration of Compound A in combination with CsA had a much greater effect than the use of CsA alone as this regimen extended survival from 13.5±1.5 days (n=2) for CsA monotherapy to greater than 21 days and 44 days.

Remarkably, the animal initially treated with Compound A alone, rescued on day 9 with steroids, and then treated with combined therapy for 43 days had vigorous graft function at day 78, 25 days after stopping CsA.

### EXAMPLE 2

### Cytokine Determination

IL1β and IL6 can be measured using the DuoSet ELISA development kits from R&D Systems (Minneapolis, Minn.), following the directions of the manufacturer. Briefly, plates are coated with capture antibody [mouse anti-human IL1β (2 μg/ml) or mouse anti-human IL6 (4 μg/ml)] and detection antibodies are used at 100 ng/ml (biotinylated goat anti-human IL1β) or 200 ng/ml (biotinylated goat anti-human IL6). Standards are prepared with recombinant protein (3.9-250 pg/ml for IL1β and 4.7-300 pg/ml for IL6). Streptavidin conjugated to horseradish peroxidase and H₂O₂/tetramethylbenzidine are used for color development. Optical density (OD) is measured at 450 nm, with correction at 570 nm.

TNFα is measured by ELISA using the mouse-anti-human TNFα antibody BC7 (Cell Sciences, Inc., Norwood, Mass.) as the capture antibody at 2.5 μg/ml, and biotinylated goat anti-human TNFα BAF210 (R&D Systems) as the detection antibody at 800 ng/ml. A standard is prepared with recombinant protein (10-10,000 pg/ml). Streptavidin conjugated to horseradish peroxidase and H₂O₂/tetramethylbenzidine is used for color development. OD is measured at 450 nm, with correction at 570 nm.

Serum samples were taken from the cynomolgus monkeys in Example 1 at various time points pre- and post-transplantation. The samples were diluted 1:2, in PBS/0.1% BSA/0.05% Tween 20. (PBS=phosphate buffered saline; BSA=bovine serum albumin.) IL6 levels (pg/ml) in pre and post heterotopic heart transplant in serum from the cynomolgus monkeys treated with Compound A were determined obtained in accordance with the above-described procedure. The results are in Table 1 as follows:

TABLE 1

| | Day 0 | Day 0 | Day | | | | |
|---|---|---|---|---|---|---|---|
| Animal | Pre | Post | 4 | Day 7 | Day 8 | Day 14 | Day 21 |
| M308 | 4.6 | 63.7 | 35.1 | 48.4 | 24.3 | | |
| M347 | 2.8 | 421.4 | 12.7 | 15 | | 6 | 7.5 |
| M376 | 2.5 | 374.9 | 11.4 | | 11 | 6.3 | 10.3 |
| M627 | 3.4 | 106.2 | 8.8 | 19.6 | | | |

M308 received Compound A monotherapy at 10 mg/kg/day b.i.d..
M347 and M376 received 5 mg/kg Compound A plus CsA tapered.
M627 received Compound A monotherapy at 10 mg/kg/day for 9.5 days, then was rescued before acute organ rejection by bolus steroids, followed by a tapered CsA regimen.

The results show that a dramatic increase in IL6 levels occurred immediately following the transplantation, followed by a substantial reduction thereafter. These results indicate that the administration of a CCR5 antagonist such as Compound A can substantially reduce or suppress the level of pro-inflammatory cytokine associated with an acute inflammatory response.

### EXAMPLE 3

### Plasma Cytokine Levels in Transplanted Cynomologous Monkeys

Serum and plasma samples taken from the cynomologous monkeys in Example 1 on the day of explant (i.e., the day of graft rejection) were analyzed using the BD Pharmingen Cytometric Bead Array Human Inflammation Kit, which can measure IL1-β, IL6, IL8, IL10, IL12p70 and TNF-α. Reagents to detect IL6, IL8 and TNF-α have been shown by the manufacturer to cross react with rhesus and cynomologus monkey proteins. At the time of the assay, reagents to measure IL1-beta and IL12p70 had not yet been tested for cross reactivity to non-human primate proteins.

Samples were assayed in the manner described in the manufacturer's protocol booklet; i.e., “Human Inflammation Kit—Instruction Manual” (BD Biosciences, Cat. No. 551811, pdf copy available at www.bdbiosciences.com). Briefly, assay standards were reconstituted with assay diluent and diluted in two fold steps by serial dilution. Capture beads were mixed and distributed into an appropriate number of 12×75 mm polystyrene tubes. Standards or samples were added to the tubes and incubated in the dark at room temperature for 1.5 hours. The incubated tubes were washed once and phycoerythrin (PE)-labeled detection antibody reagent was added. Samples were again incubated in the dark at room temperature for 1.5 hours. After one final wash, samples were resuspended in 300 uL of wash buffer. Flow cytometer data was acquired using a Becton Dickinson FACSCalibur and analyzed with Becton Dickinson CBA Software.

The results for the IL6 assay are shown in Table 2.

TABLE 2

| | | Explant | IL6 |
|---|---|---|---|
| Animal | Treatment¹ | Day | (pg/mL) |
| M308 | Compound A monotherapy at | 8 | 58 |
| | 10 mg/kg/day b.i.d. | | |
| M627 | Compound A monotherapy at | 85 | 20 |
| | 10 mg/kg/day b.i.d. | | |
| M347 | Compound A (5 mg/kg) plus tapered CsA | 44 | no sample |
| M376 | Compound A (5 mg/kg) plus tapered CsA | 21 | 22 |
| M324 | tapered CsA | 15 | 170 |
| M634 | tapered CsA | 13 | 206 |
| ¹Refer to Example 1 for a more detailed description of the treatment provided to the monkeys following surgery. Note in particular that M627 was initially treated with Compound A alone (10 mg/kg b.i.d.), was rescued on day 9 with bolus steroids, treated with combined therapy | | | |
| # for 43 days, and then from day 52 until explant received Compound A monotherapy at 10 mg/kg b.i.d. | | | |

The data in Table 2 show that IL6 cytokine levels are suppressed in monkeys treated with Compound A at the time of graft rejection relative to monkeys treated only with CsA. It is likely that the IL6 levels would have been higher in the monkeys in the absence of any treatment. The data indicate that the administration of a CCR5 antagonist such as Compound A can substantially reduce or suppress IL6 levels associated with an inflammatory response. Analogous data obtained for IL8 cytokine levels did not indicate suppression of IL8 levels for Compound A-treated monkeys relative to CsA-only treated monkeys at the time of graft rejection.

IL-12p70, TNF-alpha and IL-10 were detected at very low levels in all samples. IL-1β was detected only in some samples. There were no significant changes in the levels of these cytokines between transplanted and control monkeys or within treatment groups.

### EXAMPLE 4

### Effect of Compound A on Cytokine Expression in Human Macrophages

Human monocytes were isolated white blood cells concentrated from units of blood (i.e., leukopaks) from normal donors. To enhance CCR5 expression, the monocytes were cultured under suspension conditions in Teflon jars for 2 days in media supplemented with 12% fetal bovine serum. 10⁶ cells were seeded and allowed to attach for over 2 hours in 6-well tissue culture plates. Cells were pre-incubated with either dimethylsulfoxide (DMSO; vehicle control) or Compound A at various concentrations for 1 hour, after which RANTES (the CCR5 agonist/ligand; Pepro Tech Inc.) was added at 250 nM, or, alternatively, media was added as a control. The cells were incubated at 37° C. for 24 hours, after which the media were removed and centrifuged to precipitate the non-adherent cells. Both the adhered and non-adhered cells were lysed and subjected to mRNA isolation, and the mRNA isolate was analyzed for cytokine expression by quantitative real time PCR (TaqMan).

The relative mRNA-fold induction of cytokine gene expression in the RANTES-stimulated macrophages was calculated relative to day 2 non-stimulated, non-treated controls (i.e. fold induction=1 on day 2 for controls), as well as fold mRNA induction relative to vehicle (DMSO) (0 nM) control RANTES stimulated cells. A summary of the results of the TaqMan analysis is as follows:
(i) RANTES stimulation increased mRNA expression of IL1-beta and IL6, whereas other cytokines such as TNF-α and IFN-γ were unaffected.
(ii) Compound A was able to diminish IL1-β and IL6 up-regulation significantly.

(iii) The inhibitory effect by Compound A on IL1-β and IL6 mRNA expression in the macrophages was dose-dependent, as shown by the results in Table 3.

TABLE 3

| Compound A | IL1-beta (% | |
|---|---|---|
| (nM) | inhibition) | IL6 (% inhibition) |
| 0 | 0 | 0 |
| 3 | 37.6 (±32.1) | 43 |
| 10 | 50.1 (±25.8) | 16.1 (±6.3) |
| 30 | 59.0 (±26.8) | 65.9 (±6.3) |
| 100 | 76.8 (±21.8) | 67.1 |
| 300 | 80.6 (±12.6) | 85.9 (±12.9) |

The values are mean ± SD (if applicable). No induction of TNF-α or IFN-γ was observed so as to give percent inhibition levels.

References have been made throughout this application to various published documents in order to more fully describe the state of the art to which this invention pertains. All of these documents not previously incorporated by reference and are hereby incorporated by reference in their entireties.

While the foregoing specification teaches the principles of the present invention, with examples provided for the purpose of illustration, the practice of the invention encompasses all of the usual variations, adaptations and/or modifications that come within the scope of the following claims.

1. A method of treating or preventing stress response in a subject in need thereof, which comprises administering a therapeutically effective amount of a CCR5 antagonist to the subject.

2-3. (canceled)

4. The method according to claim 1, wherein the subject is other than a graft transplant patient.

5. The method according to claim 1, wherein the stress response is stress response to surgery.

6. The method according to claim 1, wherein the subject is a cardiac surgery patient.

7. The method according to claim 1, wherein the therapeutically effective amount of CCR5 antagonist administered to the subject is an amount effective to inhibit endogenous production of one or more pro-inflammatory cytokines selected from the group consisting of IL1 and IL6.

8-10. (canceled)

11. A method of treating or preventing hyperthermia in a subject in need thereof, which comprises administering a therapeutically effective amount of a CCR5 antagonist to the subject.

12-13. (canceled)

14. The method according to claim 11, wherein the subject is other than a graft transplant patient.

15. The method according to claim 11, wherein the stress response is surgical hyperthermia.

16. The method according to claim 11, wherein the subject is a cardiac surgery patient.

17. The method according to claim 11, wherein the therapeutically effective amount of CCR5 antagonist administered to the subject is an amount effective to inhibit endogenous production of one or more pro-inflammatory cytokines selected from the group consisting of IL1 and IL6.

18-41. (canceled)

42. A method of inhibiting endogenous production of at least one pro-inflammatory cytokine selected from the group consisting of IL1 and IL6, which comprises administering to a mammal in need of such inhibition a CCR5 modulator in an amount effective to inhibit production of the cytokine.

43. The method according to claim 42, wherein the CCR5 modulator comprises a CCR5 antagonist.

44. A method for monitoring the effectiveness of treatment of a subject suffering from an acute inflammatory response, said treatment comprising administration of a CCR5 modulator, wherein the method comprises:
(A) obtaining a pre-administration sample from the subject prior to administration of the CCR5 modulator and determining the level of expression or activity of a pro-inflammatory cytokine selected from the group consisting of IL1 and IL6 in the pre-administration sample;
(B) obtaining a post-administration sample from the subject subsequent to administration of the CCR5 modulator and determining the level of expression or activity of the pro-inflammatory cytokine; and
(C) comparing the level of cytokine expression or activity of the post-administration sample with the level of cytokine expression or activity of the pre-administration sample.

45. The method according to claim 44, which further comprises:
(D) adjusting the administration of the CCR5 modulator to increase or decrease the level of cytokine expression or activity; and
(E) repeating steps (A), (B), and (C).

46. The method according to claim 45, wherein the CCR5 modulator comprises a CCR5 antagonist.

47.-52. (canceled)
